SEQUENCE LISTING (11 sequences)

SEQ ID NO: 1 - protein, 5 aa, Artificial Sequence
  source: /note=“Description of Artificial Sequence Synthetic peptide”
SYYMN

SEQ ID NO: 2 - protein, 17 aa, Artificial Sequence
  source: /note=“Description of Artificial Sequence Synthetic peptide”
GISGDPSNTYYADSVKG

SEQ ID NO: 3 - protein, 11 aa, Artificial Sequence
  source: /note=“Description of Artificial Sequence Synthetic peptide”
DLPLVYTGFAY

SEQ ID NO: 4 - protein, 11 aa, Artificial Sequence
  source: /note=“Description of Artificial Sequence Synthetic peptide”
SGDNLRHYYVY

SEQ ID NO: 5 - protein, 7 aa, Artificial Sequence
  source: /note=“Description of Artificial Sequence Synthetic peptide”
GDSKRPS

SEQ ID NO: 6 - protein, 9 aa, Artificial Sequence
  source: /note=“Description of Artificial Sequence Synthetic peptide”
QTYTGGASL

SEQ ID NO: 7 - protein, 120 aa, Artificial Sequence
  source: /note=“Description of Artificial Sequence Synthetic polypeptide”
QVQLVESGGGLVQPGGSLRLSCAASGFTFSSYYMNWVRQAPGKGLEWVSGISGDPSNTYYADSVKGRFTISRDNSKNTLYLQMNSLRAEDTAVYYCARDLPLVYTGFAYWGQGTLVTVSS

SEQ ID NO: 8 - protein, 109 aa, Artificial Sequence
  source: /note=“Description of Artificial Sequence Synthetic polypeptide”
DIELTQPPSVSVAPGQTARISCSGDNLRHYYVYWYQQKPGQAPVLVIYGDSKRPSGIPERFSGSNSGNTATLTISGTQAEDEADYYCQTYTGGASLVFGGGTKLTVLGQ

SEQ ID NO: 9 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 10 - DNA, 360 bp, Artificial Sequence
  source: /note=“Description of Artificial Sequence Synthetic polynucleotide”
caggtgcaattggtggaaagcggcggcggcctggtgcaaccgggcggcagcctgcgtctgagctgcgcggcctccggatttaccttttcttcttattatatgaattgggtgcgccaagcccctgggaagggtctcgagtgggtgagcggtatctctggtgatcctagcaatacctattatgcggatagcgtgaaaggccgttttaccatttcacgtgataattcgaaaaacaccctgtatctgcaaatgaacagcctgcgtgcggaagatacggccgtgtattattgcgcgcgtgatcttcctcttgtttatactggttttgcttattggggccaaggcaccctggtgacggttagctca

SEQ ID NO: 11 - DNA, 327 bp, Artificial Sequence
  source: /note=“Description of Artificial Sequence Synthetic polynucleotide”
gatatcgaactgacccagccgccttcagtgagcgttgcaccaggtcagaccgcgcgtatctcgtgtagcggcgataatcttcgtcattattatgtttattggtaccagcagaaacccgggcaggcgccagttcttgtgatttatggtgattctaagcgtccctcaggcatcccggaacgctttagcggatccaacagcggcaacaccgcgaccctgaccattagcggcactcaggcggaagacgaagcggattattattgccagacttatactggtggtgcttctcttgtgtttggcggcggcacgaagttaaccgttcttggccag

(19) United States
(12) Patent Application Publication
(10) Pub. No.: US 2022/0144965 A1
(43) Pub. Date: May 12, 2022

(54) ANTI-CD38 ANTIBODIES AND PHARMACEUTICAL COMPOSITIONS THEREOF FOR THE TREATMENT OF AUTOANTIBODY-MEDIATED AUTOIMMUNE DISEASE

(71) Applicant: MORPHOSYS AG, Planegg (DE)

(72) Inventors: Daniel KLUNKER, Munich (DE); Rainer BOXHAMMER, Kolbermoor (DE); Stefan HÄRTLE, Mammendorf (DE); Stefan STEIDL, Munich (DE); Tiantom JARUTAT, Stockdorf (DE)

(21) Appl. No.: 17/434,465

(22) PCT Filed: Mar. 13, 2020
(86) PCT No.: PCT/EP2020/056757

§ 371 Date: Aug. 27, 2021

(30) Foreign Application Priority Data
Mar. 15, 2019 (EP) 19163036.7

(51) Int. Cl. C07K 16/28; A61K 39/395; A61K 45/06; A61P 37/06

(52) U.S. Cl. CPC C07K 16/2896; A61K 39/3955; A61K 45/06; A61K 2039/545; C07K 2317/732; C07K 2317/21; A61P 37/06

(57) ABSTRACT

The present invention relates to the use of an antibody or antibody fragment specific for CD38 in the prophylaxis and/or treatment of autoantibody-mediated autoimmune disease. In accordance with the present invention, an anti-CD38 antibody is effective in the treatment of anti-PLA2R positive membranous glomerulonephropathy.

### FIELD OF THE INVENTION

The present invention relates to an antibody or antibody fragment specific for CD38 useful in the treatment and/or prophylaxis of autoantibody-mediated autoimmune diseases (AD). In particular, the invention provides methods for the reduction of autoantibody titers by depletion of antibody-secreting cells using an anti-CD38 antibody alone, or in combination with one or more immunosuppressive drugs. In accordance with the present invention, an anti-CD38 antibody, alone or in combination, can be effective in the treatment and/or prophylaxis of anti-PLA2R positive membranous nephropathy (aMN). An anti-CD38 antibody includes, but is not limited to MOR202.

### BACKGROUND OF THE INVENTION

Autoimmune Diseases and Autoantibodies

Autoimmune diseases (AD) include more than 70 different disorders affecting approximately 5% of the population of the Western countries (Lleo et al. Autoimmunity Reviews 2010 Mar.; 9(5): A259-66). An AD is a clinical state caused by the activation of autoreactive T cells or autoreactive B cells or both. Certain AD are characterized by the generation of pathogenic autoantibodies. Autoantibodies are immunoglobulins that react with self-antigens. Such self-antigens may comprise proteins, nucleic acids, carbohydrates, lipids or various combinations of these and may be present in all cells (e.g. DNA) or be highly restricted to a specific cell type in one organ of the organism. In autoantibody-mediated humoral AD, autoantibodies usually occur with high titers in the sera of patients. For many AD an unambiguous and clear link of autoantibody formation, specificity and pathogenesis is proven (Suurmond and Diamond, J Clin Invest. 2015 Jun. 1; 125(6): 2194-2202). Pathogenic autoantibodies affect the disease pathway in a number of ways, including deposition of immune complexes (ICs) and inflammation, stimulation or inhibition of receptor functions, stimulation or inhibition of enzyme functions, facilitated antigen-uptake, cell lysis, microthrombosis and neutrophil activation (Ludwig et al. Front. Immunol. 2017 May; 8:603).

Systemic Lupus Erythematosus (SLE)

Systemic lupus erythematosus (SLE) for instance is a multi-gene autoimmune disorder with a prevalence of about 50 cases per 100,000 people with women more frequently affected than men. The central immunological disturbance in SLE patients is an inappropriate activation and proliferation of autoreactive memory B cells leading to an expansion of antibody secreting cells and the production of a variety of autoantibodies. The dominant self-antigens in SLE are nuclear components like DNA or ribonucleoproteins (RNPs) and the autoantibodies reactive to these antigens are of high-affinity, somatically mutated and of the IgG isotype. SLE patients show high levels of serum antinuclear antibodies (ANAs). Autoantibodies to cytoplasmic antigens, cell membrane antigens, phospholipid-associated antigens, blood cells, endothelial cells, nervous system antigens, plasma proteins, matrix proteins, and miscellaneous antigens may also be present (FIG. 12). In SLE, many of these autoantibodies lead to the formation of ICs that appear to be directly pathogenic following deposition in several tissues.

Treatment options for SLE comprise antimalarial medicament, steroidal and non-steroidal anti-inflammatory agents, immunosuppressive drugs (including cyclophosphamide (CTX), azathioprine (AZA), mycophenolic acid (MMF) and methotrexate (MTX)), as well as immune cell targeted therapies (Yildirim-Toruner C, Allergy Clin Immunol. 2011 February; 127(2):303-12). These immunosuppressive or cytotoxic drugs and anti-CD20-mediated B cell depletion can induce remissions in patients with SLE. However, current treatment protocols frequently fail to prevent relapses (Stichweh, D. Curr. Opin. Rheumatol. 2004 16:577-587.5).

Graves' Disease (Morbus Basedow)

Graves' Disease also known as toxic diffuse goiter, is an autoimmune disease that affects the thyroid. Grave's disease will develop in about 0.5% of males and 3% of females (Burch HB, Cooper DS, 2015, JAMA 314 (23): 2544-54). It frequently results in and is the most common cause of hyperthyroidism in the United States (about 50 to 80% of cases). Symptoms of hyperthyroidism may include irritability, muscle weakness, sleeping problems, a fast heartbeat, poor tolerance of heat, diarrhea, unintentional weight loss, thickening of the skin on the shins, known as pretibial myxedema, and eye bulging, a condition caused by Graves' ophthalmopathy. The direct cause of Graves' disease are autoantibodies directed against the receptor for thyroid-stimulating hormone (thyroid-stimulating hormone receptor (TSHR)). Autoantibodies to thyroglobulin and to the thyroid hormones T3 and T4 may also be produced. TSHR autoantibodies mimic TSH and activate TSHR in an unregulated manner, thereby causing hyperthyroidism. The treatment options for Graves' disease include antithyroid (thionamide) drugs, thyroid ablation by radioiodine, and surgery (thyroidectomy).

The challenge in treating Graves' Disease remains however, to inhibit the development or ongoing production of TSHR autoantibodies.

Myasthenia Gravis (MG)

Myasthenia Gravis (MG) affects 50 to 200 per million people. It is newly diagnosed in three to 30 per million people each year. MG is a long-term neuromuscular AD that leads to varying degrees of skeletal muscle weakness and abnormal fatigability and is caused by the presence of autoantibodies reactive to components of the postsynaptic muscle endplate localized at the neuromuscular junction (unction between nerve and muscle). In particular, these autoantibodies block or destroy nicotinic acetylcholine receptors, which in turn prevents nerve impulses from triggering muscle contractions. Other autoantibodies are found against a related protein called MuSK, a muscle-specific kinase and LRP4, Agrin and titin proteins. Generally, MG is treated with drugs known as acetytcholinesterase inhibitors such as neostigmine and pyridostigmine. Immunosuppressants, such as prednisone or azathioprine are also often used. In certain cases, the surgical removal of the thymus may improve symptoms of the disease. Plasmapheresis and high dose intravenous immunoglobulin (IVIG) may be used during sudden flares of the condition to remove putative autoantibodies from the circulation or to dilute and bind the circulating antibodies, respectively. Both of these treatments have relatively short-lived benefits, typically measured in weeks, and often are associated with high costs. If the breathing muscles become significantly weak, mechanical ventilation may be required.

Anti-PLA2R Positive Membranous Glomerulonephritis (aMN)

Anti-PLA2R-autoantibody-mediated membranous nephropathy (aMN), historically often referred to as Idiopathic Membranous Glomerulonephritis or idiopathic membranous nephropathy (IMN) is a primary membranous nephropathy and the leading cause of nephrotic syndrome in adults (Ronco P, Debiec H Lancet. 2015 May 16; 385(9981):1983-92). About 80% of membranous nephropathies are idiopathic, while 20% are related to other diseases or exposures. The overall global incidence is estimated at 1.2 per 100,000 per year. Although the disease usually progresses slowly, approximately 30% to 40% of patients eventually develop End Stage Renal Disease. Patients with MN remaining nephrotic are at increased risk for thromboembolic and cardiovascular events. However, although not all aspects of the pathogenesis of MN are understood, the disease can no longer be considered idiopathic. M-type phospholipase A2 receptor (PLA2R), a transmembrane protein expressed on podocytes, has been defined as the major autoantigen of MN (Beck L H Jr et al. N Engl J Med. 2009 Jul. 2; 361(1):11-21). Autoantibodies binding to the PLA2R antigen are highly specific to primary MN. Recent investigations revealed the presence of anti-PLA2R autoantibodies in approximately 75% of patients with IMN that considerably correlate with disease activity (Bomback A S, Clin J Am Soc Nephrol. 2018 May 7; 13(5):784-786). The fact that the disease defining glomerular basement changes contain both PLA2R protein as well as antibody complex deposits provides evidence that anti-PLA2R antibodies play a major causative role in MN. An additional 5% of patients who are negative for anti-PLA2R antibodies have antibodies against another podocyte antigen—the thrombospondin type-1 domain-containing 7A (Tomas N M et al. N Engl J Med 2014; 371: 2277-2287). In rare neonatal MN cases, neutral endopeptidase (NEP) located on the foot process membrane of the podocytes and the brush border of renal tubules has been identified as the relevant antigen (Ronco P et al. J Am Soc Nephrol. (2005) 16:1205-13. Taken together, about 80% of patients with IMN have antibodies directed against a specific, identifiable podocyte antigen. Symptoms of membranous nephropathy include, but are not limited to swelling in the legs and ankles, increased protein in urine, edema, hypoalbuminemia, elevated serum lipids, in particular high cholesterol. Thus, autoimmune membranous nephropathy is an immune-mediated glomerular disease that is characterized by the presence of anti-PLA2R autoantibodies and/or anti-THSD7A autoantibodies. In neonatal autoimmune MN, autoantibodies against NEP are present which were transferred from the mother.

At present, there is no approved standard treatment for MN. The current treatment regimen mainly comprises off-label use of various non-immunosuppressive and immunosuppressive drugs. Patients diagnosed with MN and proteinuria >3.5 g per day initially receive supportive therapy with a combination of Angiotensin-converting enzyme inhibitors (ACEi) or Angiotensin II Receptor blockers (ARB), statins and diuretics as per current clinical standard. If not responding with a significant decrease of proteinuria within months, escalation to immunosuppressive therapy (IST) is indicated. Immunosuppressive therapies include corticosteroids alternating with alkylating agents (e.g. cyclophosphamide), and calcineurin inhibitors (CNIs, e.g. Cyclosporin A, tacrolimus (FK506)), Mycophenolat-Mofetil (MMF) or Rituximab even though none of these drugs is approved for use in MN. To a lesser extent, adrenocorticotropin (ACTH) has been used. Treatment effects of these drug combinations appear to be similar: remission of proteinuria can be expected in about 50 to 60% of patients in the first year and in about 70 to 80% at 2 to 3 years in comparison to the remission rate of about 30% in controls treated with supportive care only (spontaneous remission).

Out of all patients with primary membranous nephropathy not receiving IST, 30% to 40% progress to end stage renal disease in 10 years after disease onset. IST reduces the progression rate to 10% or less. Relapses in proteinuria are seen in about 25% of patients previously treated with IST. The cases are usually re-treated with a different IST combination. A drawback of the ISTs described above is that they exhibit a considerable degree of toxicity and are associated with significant adverse effects and a high relapse rate. 25% of patients treated with cyclophosphamide demonstrate adverse events, which include infection, infertility, hematologic toxicity and malignancy later in life. Disadvantages of CNIs include long-term nephrotoxicity, the need to closely monitor drug levels and the increased risk for hypertension and diabetes. Relapse rates with calcineurin inhibitors seem to be higher than with cyclophosphamide (40-50% versus 25%). Due to considerable evidence showing that anti-PLA2R antibodies correlate with disease activity previously established therapy algorithms are changing.

Recently introduced off-label use therapy with anti-CD20 therapeutic antibody rituximab allows for a more specific IST approach by depleting B-cell populations involved as progenitors in producing the causative anti-PLA2R autoantibodies. Rituximab response rates seem to be similar to alkylating agents and CNIs, whereas side effects seem to be less than for other drugs used in IST. However, CD20, the target of rituximab is not present on mature long-lived antibody-secreting plasma cells (that are the main source of endogenous immunoglobulins). On early plasmablasts there is only minor residual CD20 expression, compared to the CD20 expression on mature B cells. This is a possible explanation for the sub-optimal efficacy of rituximab therapy in MN patients with high anti-PLA2R antibody titers. In this respect, direct targeting of plasmablasts as well as plasma cells should lead to a more pronounced reduction in immunoglobulins in general, and therefore also on a reduction of autoantibodies. A substantial portion of the anti-PLA2R antibodies in aMN is possibly produced by a long-lived plasma cell pool with a CD20 negative, but CD38 positive immunophenotype, that is not dependent on continuous replenishment of differentiating B-cells. Thus, a direct plasma cell targeting strategy might have a more profound effect on suppression of pathogenic autoantibodies. In particular, this is important for patients with inadequate response to rituximab (anti-CD20) therapy that maintain high levels of autoantibody titers despite B-cell depletion.

Pemphigus

Pemphigus vulgaris is an autoimmune intra-epidermal muco-cutaneous disorder of the skin and mouth resulting in blister formation. Lesions occur with an increased incidence of 0.5 to 3.2 cases per 100,000 people every year. These lesions predominantly occur between age 40 to 60 with equal gender predilection. Pemphigus patients present with circulating autoantibodies against pemphigus antigens (desmoglein 3, desmoglein 1, desmocollins, plakoglobin) on epithelial keratinocytes. Disruption of these antigens by the antigen-autoantibody reaction has a marked effect on the integrity of the epidermis resulting in cellular detachment (acantholysis), suprabasilar clefting and subsequent bullae formation. Binding of the autoantibodies to keratinocytes also results in release of protease and plasminogen activator (converts plasminogen to plasmin) from the cells further amplifying acantholysis. Treatment options for high-grade lesions include systemic glucocorticoids and combinations of corticosteroids, immunosuppressive agents, pulse therapy, photophoresis and plasmaphoresis.

Sjögren's Syndrome

Sjögren's syndrome is a systemic autoimmune disease characterized by focal infiltration of lymphocytes into the exocrine glands and lacrimal glands resulting in dry mouth (xerostomia) and dry eyes (keratoconjunctivitis sicca), respectively. In Sjögren's syndrome, the presence of lesions are associated with chronic inflammatory infiltrates with release of autoantibodies against the salivary glandular epithelial cells. Other autoantibodies in Sjögren's syndrome are directed against ribonucleoprotein autoantigens Ro/SS-A and La/SS-B, coiled-coil-containing molecules, members of golgin family, poly (ADP) ribose polymerase (PARP) and type 3 muscaranic receptor. At present, a targeted treatment of Sjögren's syndrome is not available and current therapeutic approaches are only symptomatic by treating the sicca and fatigue symptoms, for example with pilocarpine, bromhexine and hydroxychloroquine, respectively.

Anti-NMDA Encephalitis

The most common antibody-mediated acute autoimmune encephalitis is the anti-N-methyl-D-aspartate-receptor (NMDAR) encephalitis (Granerod J et al. Lancet Infect Dis 2010, 10:835-44). Its incidence is estimated at 3-5 per 1,000,000 population and year. Anti-NMDA encephalitis represents a model disease for a group of syndromes characterized by detection of autoantibodies targeting synaptic structures. Anti-NMDAR antibodies are most common, followed by antibodies against leucine-rich glioma inactivated-1 (LGI1) The contactin-associated protein like 2 (Caspr2), α-amino-3-hydroxy-5-methyl-4-isoxazolepropionic acid receptor (AMPAR), gamma-aminobutyric acid (GABA)-A and -B receptors, dipeptidyl-peptidase-like protein-6 (DPPX), and glycine receptor (GlyR) antibodies are other examples of neuronal cell-surface antibodies. Anti-NMDAR encephalitis preferentially occurs in young adults and children, predominantly women (80%). Approximately 70% of the patients develop prodromal symptoms (e.g. headache, fever, rapid change of behavior, anxiety, hallucinations, and psychosis). Abnormal movements (e.g. orofacial dyskinesias, chorea, and stereotyped movements) and decrease of consciousness, coma, and severe global autonomic dysregulation (sometimes leading to hypoventilation and asystolia) ensue. Seizures and status epilepticus may occur at any stage of the disease. Approximately, 50% of patients respond well to IVIGs, steroids, or plasma exchange and the other 50% require rituximab alone or in combination with cyclophosphamide. However, in some patients, recovery is incomplete, may take years, and mortality due to intensive care complications can be as high as 7%

The presence of pathogenic autoantibodies in the autoantibody-mediated autoimmune diseases exemplified above is a consequence of a failure or breakdown of central and/or peripheral B cell tolerance toward the corresponding self-antigens.

Central and Peripheral B Cell Tolerance

B cell development starts in the bone marrow. There, the nascent membrane-bound B cell receptor (BCR) repertoire is generated by somatic recombination of immunoglobulin heavy- and light chain gene segments. The downside of producing this huge variety in the early BCR repertoire by random somatic V(D)J recombination is a concurrent generation of autoantibodies that might have a potential of being pathogenic. At least three mechanism exist to prevent the development of autoimmunity. First, self-reactive B cells are deleted by apoptosis. Second, auto-reactive B cells lower the self-reactive affinities of their BCRs through changes of the VL domains by secondary Ig light chain recombination, a process referred to as receptor editing. The third mechanism to silence self-reactive B cells is anergy, which makes such cells unresponsive to antigen. These central tolerance mechanisms take place in the bone marrow. Thus, autoreactivity of the emerging antibody repertoire is prevented by apoptosis, receptor editing, and induction of anergy in B cells expressing autoreactive antibodies (Wardemann and Nussenzweig, Adv Immunol. 2007; 95:83-110).

During B cell differentiation, transitional B cells emerging from the bone marrow continue to mature in peripheral lymphoid organs (e.g. in the spleen, lymph nodes), where additional, peripheral tolerance mechanisms are in place. The exact mechanisms of peripheral tolerance are still under investigation, but ligand (antigen) recognition by the BCR, similar to the central tolerance checkpoints in the bone marrow, is involved. They also may involve controlled migration and limited availability of BAFF, CD22, Siglec-G, miRNA and follicular regulatory T cells (Tregs).

The end-stage products of B cell differentiation are antibody-secreting plasma cells. Upon activation by antigen, mature naïve B cells either develop directly (T cell independent) into antibody-secreting cells or differentiate during T cell dependent immune responses in the germinal center via proliferating pre-plasmablasts and plasmablasts into sessile non-dividing plasma cells or memory B cells. Both plasmablasts and plasma cells produce and secrete antibodies and thereby provide humoral immunity. When they are derived from self-reactive B cells, plasmablasts and plasma cells contribute to autoantibody production (Hiepe and Radbruch, Nat Rev Nephrol. 2016 April; 12(4):232-40).

The failure of one or more of the central and/or peripheral tolerance mechanisms leads to an increased number of circulating self-reactive B cells (i.e. autoantibody expressing B cells) and self-reactive plasmablasts and plasma cells (i.e. autoantibody expressing and secreting cells) favoring the development of autoantibody-mediated AD. Once the production of autoantibodies has started, their production level is maintained either by continued activation of autoreactive B cells resulting in a continuous formation of short-lived plasma cells or through the formation of long-lived plasma cells, or both (Manz R A et al, Annu Rev Immunol (2005) 23:367-86).

As autoantibodies are often the underlying cause of autoimmune pathology, B cells, plasmablasts and plasma cells are promising therapeutic targets in AD. Short-lived plasma cells respond to conventional immunosuppressive drugs that directly inhibit proliferating plasmablasts and B cells. Non-proliferating short-lived plasma cells disappear within a few days of initiating these therapies, as they are no longer being replenished. Therapies that target B cells, such as anti-CD20 (rituximab) and anti-BAFF (belimumab) (see for example WO2002002641, WO2009052293A1) reduce the levels of B cells in patients in need of such reduction and therefore attenuate the generation of short-lived plasmablasts and plasma cells but such therapies do not affect the long-lived memory plasma cell compartment. In cases in which autoantibody production is not affected by this therapeutic strategy, it should be considered that the autoantibodies are potentially being secreted by long-lived memory plasma cells. Moreover, it can be assumed that blockade of factors or cells that stimulate autoreactive B cells, for example by targeting type I interferon (IFN), TH cells or regulatory T (Treg) cells, will prevent the development of short-lived plasmablasts and plasma cells, but not plasma-cell memory.

In humans, the long-lived bone marrow-derived plasma cell population is phenotypically defined as CD19−, CD38hi, CD138+(Halliley J L et al. Immunity. 2015 Jul. 21; 43(1):132-45). CD20, a well-known common pan-B cell marker is usually not expressed on human plasmablasts (Ellebedy A H et al. Nat Immunol. 2016 Oct.; 17(10):1226-34) or long-lived human plasma cells (Halliley J L et al. Immunity. 2015 Jul. 21; 43(1):132-45). Potential mechanisms underlying the maintenance of long-term antibody responses can be generally divided into memory B cell-dependent and memory B cell-independent models. In a rhesus animal model, it has been shown that after surgically removing potential B cell reservoirs from solid tissues (e.g. spleen and lymph nodes), as well as depletion of all detectable tetanus-specific memory B cells from the circulation using an anti-CD20 antibody, tetanus-specific serum antibody titers continued to be maintained above the protective threshold for the lifespan of the immune host with decay rate kinetics that were indistinguishable from untreated controls (Hammarlund E et al. Nat Commun. 2017; 8: 1781). Thus, antibody responses following tetanus vaccination are long-lived and provide lifelong protective immunity against this disease. Further analysis of tetanus-specific plasma cells revealed that 10 years after immunization, long-lived vaccine-induced plasma cells were preferentially identified in certain bone marrow compartments. Altogether, these studies provide a framework in which the maintenance of long-term serum antibody responses appears to be maintained by long-lived plasma cells independently of memory B cells.

As described above current treatment options of AD include systemic immunosuppression (i.e. with high doses of corticosteroids, such as dexamethasone). The cytotoxic drug cyclophosphamid (Endoxan®) has been shown to suppress T-helper cell functions with prolonged reduction of B cells due to the slower rate of recovery of B lymphocytes from an alkylating agent and thereby cyclophosphamid suppresses B cell activation. Further immunosuppressive drugs include, but are not limited to azathioprine, mycophenolic acid and methotrexate. Proteasome inhibitors, such as bortezomib have been shown to deplete short-lived and long-lived plasma cells and first clinical trials using bortezomib for the treatment of SLE and thrombotic thrombocytopenic purpura are promising (Alexander T et al. Ann Rheum Dis (2015) 74:1474-8; Patriquin et al. Br J Haematol (2016) 173: 779-85).

WO2012092612 discloses anti-CD38 antibodies and alleges their possible therapeutic use for a plethora of autoimmune diseases. In fact, WO2012092812 determines the anti-tetanus response in a HuScid mouse model and shows experiments performed with a surrogate murine anti-CD38 antibody in a collagen induced arthritis and SLE autoimmune mouse model only. WO2012092612 is silent about determining antibody titers in human samples after anti-CD38 therapy and does not mention or shows any data on anti-PLA2R positive membranous nephropathy to be treated with an anti-CD38 antibody.

Schuetz C et al. (Blood Adv. 2018; 2(19):2550-2553) describe the use of the anti-CD38 antibody daratumumab for the treatment of autoimmune hemolytic anemia. Cole S et al. Arthritis Res Ther. 2018; 20(1):85 evaluate the potential of daratumumab in the treatment of patients with RA and SLE.

Beck L H et al. (J Am Soc Nephrol. 2011; 22(8):1543-50) disclose the use of the anti-CD20 antibody rituximab to deplete B cells in patients with idiopathic membranous nephropathy. This disclosure does not teach or suggest the depletion of plasma cells with an anti-CD38 antibody in these patients.

Nevertheless, patients with AD still suffer from high morbidity and increased mortality. In spite of the progress in the development of novel anti-autoimmune agents (such as bortezomib), many autoantibody-mediated ADs that most probably involve CD38 positive autoantibody-secreting cells, still have a poor prognosis. All of the above mentioned treatment options have drawbacks, side effects, or their use is limited to certain types of patient groups.

Thus, there is still a high and unmet medical need for novel and improved treatment methods for patients suffering from autoantibody-mediated AD.

The present inventors have identified that CD38 represents an excellent and valid antigen to directly target antibody-secreting cells such as plasmablasts and plasma cells in autoantibody-mediated autoimmune disorders (e.g.: SLE, aMN). First, CD38 shows a very high expression on plasmablasts and plasma cells (FIG. 4) and second, CD38 has no or a significant lower expression on other cells types compared to plasmablasts and plasma cells. Using an anti-CD38 antibody therefore allows targeting the source of pathogenic autoantibodies as a sustainable therapeutic approach with potentially long-lasting effects due to the elimination of short- and long-lived plasma cells. In essence, such targeting can be generalized as follows: antibodies specific to the CD38 surface antigen of antibody-secreting cells are administered to a patient. These anti-CD38 antibodies specifically bind to the CD38 antigen of both antibody-secreting cells producing normal antibodies and pathogenic autoantibodies. The antibody bound to the CD38 surface antigen then leads to the destruction and depletion of these cells. Irrespective of the approach, the main goal is to diminish the cells producing the autoantibodies.

Endogenous Anti-Tetanus Antibody Titer as Marker to Evaluate the Impact of MOR202 on Plasma Cell Function

Longitudinal studies in mice (Manz R A, et al. Nature. 1997; 388: 133-134) and man (Hammarlund E et al. Nat Med. 2003 September; 9(9):1131-7) highlight the advantages of inducing and maintaining effective serum concentrations of antibodies (antibody titers) that persist and remain protective for long time periods, up to the lifetime of the immune system. Protective humoral immunity is conferred by stable titers of specific antibodies for example generated by routine vaccinations against e.g. measles, mumps, tetanus, diphtheria, or smallpox. Plasma cells and their immediate precursors are known as the cellular basis of this humoral immunity and as serum-specific antibody titers are valuable markers of the humoral arm they may be used as indicator for the presence and/or activity of plasma cells producing these antibodies. Mouse studies using anti-CD20 treatment to deplete naïve and memory B cells showed that loss of B cells did not significantly impact the plasma cell pool, even after a long period of time (Ahuja A et al. Proc Natl Acad Sci USA. 2008 Mar. 25; 105(12):4802-7). Similarly, humans undergoing B cell ablation therapies maintain serum antibody titers to common antigens for at least one year (Cambridge G et al. Arthritis Rheum. 2006 March; 54(3):723-32.). Thus, these reports indicate that (long-lived) plasma cells are the essential components of lasting humoral memory in mice and humans. It is well established that plasma cells can persist for extended periods even without input from recently activated naïve or memory B cells. Here, the inventors show for the first time, that MOR202 administration leads to a decrease of endogenous anti-tetanus toxoid antibody titers in human subjects and describe in the examples how treatment of autoantibody-mediated membranous nephropathy, in particular anti-PLAR2 positive autoimmune MN, with MOR202 is put into practice.

### SUMMARY OF THE INVENTION

The present invention provides antibodies or antibody fragments specific for CD38, for use in the treatment and/or prevention of autoantibody-mediated autoimmune diseases and related conditions. In particular, the anti-CD38 antibody or antibody fragment is for use in the treatment and/or prevention of idiopathic membranous glomerulonephritis. Preferably, the anti-CD38 antibody or antibody fragment is for use in the treatment and/or prevention of anti-PLA2R positive membranous glomerulonephritis. In some aspects, the anti-CD38 antibody or antibody fragment is for use in the treatment and/or prevention of systemic lupus erythematosus (SLE).

Furthermore, the present invention provides pharmaceutical compositions comprising a therapeutically effective amount of an antibody or antibody fragment specific for CD38 for use in the treatment and/or prevention of autoantibody-mediated autoimmune diseases. In particular, the anti-CD38 antibody or antibody fragment of the pharmaceutical composition is for use in the treatment and/or prevention of idiopathic membranous glomerulonephritis. Preferably, the anti-CD38 antibody or antibody fragment of the pharmaceutical composition is for use in the treatment and/or prevention of anti-PLA2R positive membranous glomerulonephritis. In some aspects, the anti-CD38 antibody or antibody fragment of the pharmaceutical composition is for use in the treatment and/or prevention of systemic lupus erythematosus (SLE).

MOR202, a monoclonal human anti-CD38 antibody, targets antibody-secreting cells such as plasmablasts and plasma cells primarily via antibody-dependent cell-mediated cytotoxicity (ADCC) and antibody-dependent cell-mediated phagocytosis (ADCP). During a clinical trial with MOR202, efficient killing of tumorous plasma cells (i.e. multiple myeloma cells) as well as benign plasma cells has been demonstrated. In patients suffering from multiple myeloma (MM), plasma cell depletion by MOR202 leads to a significant reduction in M-Protein. The M-Protein, also known as M component, M spike, spike protein, paraprotein or myeloma protein, is an immunoglobulin (antibody) or a fragment thereof secreted by a malignant, tumorous plasma cell clone. Due to the abnormal monoclonal proliferation of the malignant plasma cells in MM, the M-Protein is produced in vast excess, which leads to a multitude of deleterious effects on the body characteristic for MM (e.g. impaired immune function, abnormally high blood viscosity and kidney damage). MOR202 is effective in depleting plasma cells that are the source of the M-Protein, consequently leading to a decrease of M-Protein titers.

The effect of MOR202 on plasma cells is shown by assessment of the anti-Tetanus Toxoid (anti-TT) antibody titer in the serum as marker for depletion of specific plasma cells. After MOR202 administration, serum anti-TT antibody levels were significantly reduced as compared to the baseline prior to MOR202 administration.

Overall, the present inventors demonstrate that MOR202 efficiently reduces malignant (M-Protein) and/or protective antibody (anti-TT) levels in human serum, indicating a long-term depletion of plasmablasts and plasma cells. In contrast to other anti-CD38 antibodies, MOR202 is expected to spare cells with low CD38 expression (e.g. NK cells) and therefore offers an optimal safety profile.

This observed effect of MOR202 on the reduction of serum antibody titers is new, and the prior art does not teach, suggest or provide any rational for using MOR202 for the treatment of autoantibody-mediated AD.

In a particular aspect of the invention, the antibody or antibody fragment comprises a HCDR1 region of amino acid sequence SEQ ID NO: 1, a HCDR2 region of amino acid sequence SEQ ID NO: 2, a HCDR3 region of amino acid sequence of SEQ ID NO: 3, a LCDR1 region of amino acid sequence SEQ ID NO: 4, a LCDR2 region of amino acid sequence of SEQ ID NO: 5 and a LCDR3 region of amino acid sequence SEQ ID NO: 6 for use in the treatment and/or prevention of autoantibody-mediated autoimmune diseases, in particular for use in the treatment and/or prevention of systemic lupus erythematosus (SLE), or idiopathic membranous glomerulonephritis, preferably for use in the treatment and/or prevention of anti-PLA2R positive membranous glomerulonephritis.

The present disclosure also provides pharmaceutical compositions comprising an antibody or antibody fragment specific for CD38, and a suitable pharmaceutical carrier, excipient or diluent for use in the prophylaxis and/or treatment of autoantibody-mediated autoimmune diseases.

In a further particular aspect, the pharmaceutical compositions may additionally comprise further therapeutically active ingredients suitable for use in combination with the antibody or antibody fragments of the invention. In a more particular aspect, the further therapeutically active ingredient is an agent for the treatment of autoantibody-mediated autoimmune diseases.

In one aspect of the invention, this invention provides a method for the prophylaxis and/or treatment of autoantibody-mediated AD in a subject in need thereof, in particular humans, which method comprises administering an effective amount of a pharmaceutical composition, comprising an anti-CD38 antibody or antibody fragment to said subject.

The invention also provides a method for the prophylaxis and/or treatment of idiopathic membranous glomerulonephritis (IMN) in a subject in need thereof, said method comprising the step of administering an effective amount of a pharmaceutical composition, comprising an anti-CD38 antibody or antibody fragment to said subject.

In particular, the invention provides a method for the prophylaxis and/or treatment of anti-PLA2R positive membranous glomerulonephritis (aMN) in a subject in need thereof, said method comprising the step of administering an effective amount of a pharmaceutical composition, comprising an anti-CD38 antibody or antibody fragment to said subject.

In one aspect, this invention provides a method for the prophylaxis and/or treatment of systemic lupus erythematosus (SLE) in a subject in need thereof, said method comprising the step of administering an effective amount of a pharmaceutical composition, comprising an anti-CD38 antibody or antibody fragment to said subject.

In one aspect, this invention provides an antibody, or antibody fragment, specific for CD38 for use in the prophylaxis and/or treatment of autoantibody-mediated AD in a mammal, in particular humans, afflicted with said autoimmune disease.

Other objects and advantages will become apparent to those skilled in the art from a consideration of the ensuing detailed description.

Moreover, the antibodies or antibody fragments, specific for CD38, useful in the pharmaceutical compositions and treatment methods disclosed herein, are pharmaceutically acceptable as prepared and used.

### BRIEF DESCRIPTION OF THE DRAWINGS

FIG. 1 shows schematically the main cell types of B cell differentiation and the level of CD19, CD20 and CD38 expression. CD38 expression during B-cell ontogeny is tightly regulated: CD38 is present on bone marrow precursor B cells but is lost on mature B cells. On germinal center B cells, CD38 protects against apoptosis, but on leaving the germinal center, memory B cells lack or have only reduced levels of the antigen. On terminally differentiated short and long-lived plasma cells that are antibody-secreting cells, CD38 is one of the few surface antigens present and is highly expressed (Hamblin T J, Blood 2003 102:1939-1940).

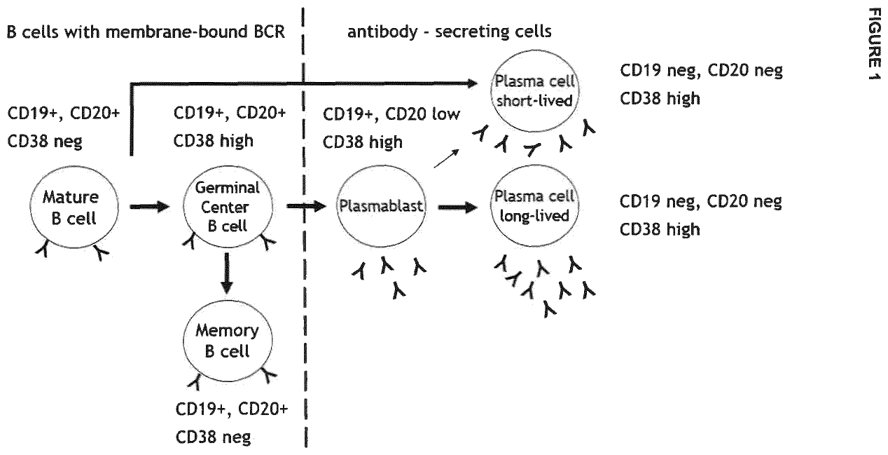

FIG. 2 shows schematically the main B cell types that are targeted by anti-CD20 B cell depleting antibody therapies (e.g. treatment with Rituximab).

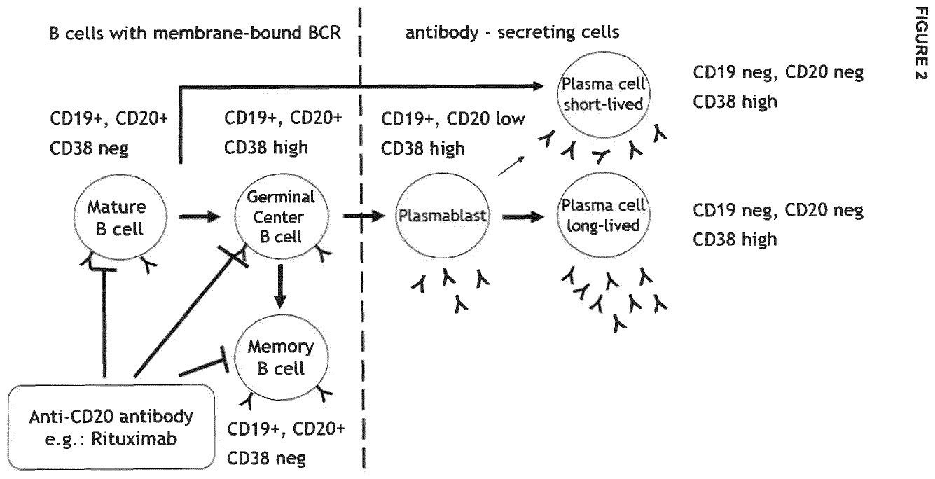

FIG. 3 shows schematically the main antibody-secreting cell types that are targeted by anti-CD38 antibody therapies (e.g. treatment with MOR202).

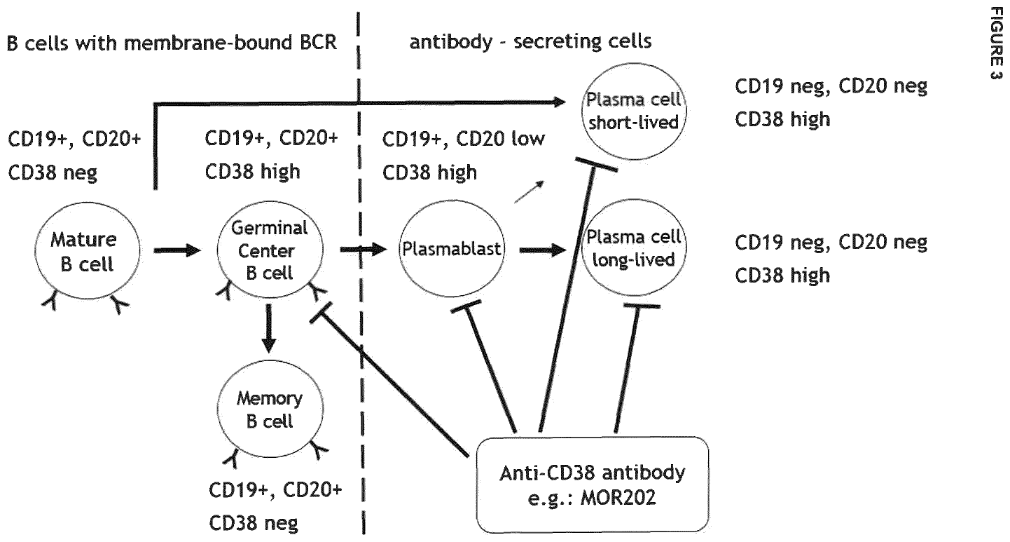

FIG. 4 shows high CD38 expression on plasma cells of healthy individuals and patients with multiple myeloma determined by FACS.

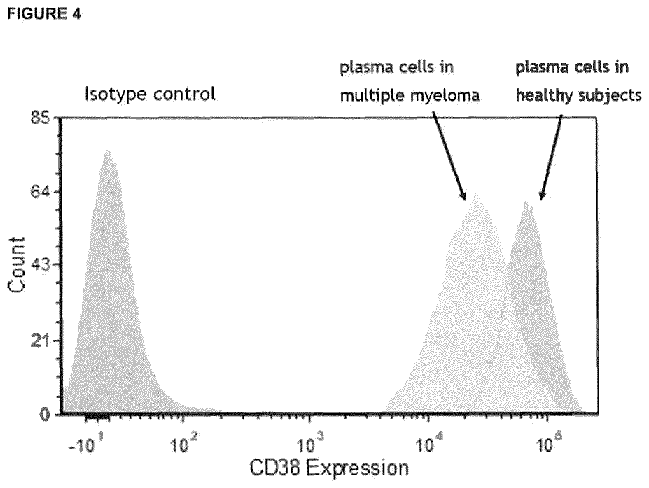

FIG. 5 shows the change (in %) of anti-tetanus toxoid (anti-TT) antibody titers in subjects post MOR202 administration at day 15 of cycle 1 (i.e. 2 weeks after start of MOR202 treatment) compared to baseline.

FIG. 6 shows the change (in %) of anti-tetanus toxoid (anti-TT) antibody titers in subjects post MOR202 administration at day 15 of cycle 2 (i.e. 6 weeks after start of MOR202 treatment) compared to baseline.

FIG. 7 shows the change (in %) of M-Protein levels in the patient cohort treated once weekly with MOR202 in combination with dexamethasone compared to baseline (best response).

FIG. 8 shows the change (in %) of M-Protein levels in the patient cohort treated once weekly with MOR202 in combination with lenalidomide/dexamethasone compared to baseline (best response).

FIG. 9 shows the change (in %) of M-Protein levels in the patient cohort treated once weekly with MOR202 in combination with pomalidomide/dexamethasone compared to baseline (best response).

FIG. 10 shows specific killing of a CD38 high expressing multiple myeloma plasma cell line by MOR202 while sparing CD38 low expressing NK cells compared to the anti-CD38 antibodies daratumumab (Dara) and isatuximab.

FIG. 11 shows the clinical trial schedule MOR202 tested in subjects with aMN.

FIG. 12 exemplifies various autoantibodies that can be detected in patients with systemic lupus erythematosus (SLE).

### DETAILED DESCRIPTION OF THE INVENTION

### Definitions

The following terms are intended to have the meanings presented therewith below and are useful in understanding the description and intended scope of the present invention.

When describing the invention, which may include antibodies, antibody fragments, pharmaceutical compositions comprising such antibodies or antibody fragments, and methods of using such antibodies, antibody fragments and compositions, the following terms, if present, have the following meanings unless otherwise indicated.

The articles ‘a’ and ‘an’ may be used herein to refer to one or to more than one (i.e. at least one) of the grammatical objects of the article. By way of example ‘an analogue’ means one analogue or more than one analogue.

The term “CD38” refers to a protein known as CD38, having the following synonyms: ADP-ribosyl cyclase 1, cADPr hydrolase 1, Cyclic ADP-ribose hydrolase 1, T10.

Human CD38 (UniProt P28907) has the following amino acid sequence:

(SEQ ID NO.: 9)
MANCEFSPVSGDKPCCRLSRRAQLCLGVSILVLILVVVLA
VVVPRWRQQWSGPGTTKRFPETVLARCVKYTEIHPEMRHV
DCQSVWDAFKGAFISKHPCNITEEDYQPLMKLGTQTVPCN
KILLWSRIKDLAHQFTQVQRDMFTLEDTLLGYLADDLTWC
GEFNTSKINYQSCPDWRKDCSNNPVSVFWKTVSRRFAEAA
CDVVHVMLNGSRSKIFDKNSTFGSVEVHNLQPEKVQTLEA
WVIHGGREDSRDLCQDPTIKELESIISKRNIQFSCKNIYR
PDKFLQCVKNPEDSSCTSEI

CD38 is a type II transmembrane glycoprotein and an example of an antigen that is highly expressed on antibody-secreting cells (including autoantibody-secreting plasmablasts and plasma cells). Functions ascribed to CD38 include both receptor-mediated adhesion and signaling events and (ecto-) enzymatic activity. As an ectoenzyme, CD38 uses NAD+ as substrate for the formation of cyclic ADP-ribose (cADPR) and ADPR, but also of nicotinamide and nicotinic acid-adenine dinucleotide phosphate (NAADP). cADPR and NAADP have been shown to act as second messengers for Ca2+ mobilization. By converting NAD+ to cADPR, CD38 regulates the extracellular NAD+ concentration and hence cell survival by modulation of NAD-induced cell death (NCID). In addition to signaling via Ca2+, CD38 signaling occurs via cross-talk with antigen-receptor complexes on T and B cells or other types of receptor complexes, e.g. MHC molecules, and is in this way involved in several cellular responses, but also in switching and secretion of IgG antibodies.

The term “anti-CD38 antibody”, as used herein, includes anti-CD38 binding molecules in its broadest sense; any molecule which specifically binds to CD38 or inhibits the activity or function of CD38, or which by any other way exerts a therapeutic effect on CD38 is included. Any molecule that interferes or inhibits CD38 functionality is included. The term “anti-CD38 antibody” includes, but is not limited to, antibodies specifically binding to CD38, alternative protein scaffolds (e.g.: fibronectin scaffolds, ankyrins, maxybodies/avimers, protein A-derived molecules, anticalins, affilins, protein epitope mimetics (PEMs) or the like) binding to CD38, nucleic acids (including aptamers) specific for CD38 or small organic molecules specific for CD38.

Antibodies specific for CD38 are described for example in WO199962526 (Mayo Foundation); WO200206347 (Crucell Holland); US2002164788 (Jonathan Ellis) which is incorporated by reference in its entirety; WO2005103083 (MorphoSys AG), U.S. Ser. No. 10/588,568, which is incorporated by reference in its entirety, WO2006125640 (MorphoSys AG), U.S. Ser. No. 11/920,830, which is incorporated by reference in its entirety, and WO2007042309 (MorphoSys AG), U.S. Ser. No. 12/089,806, which is incorporated by reference in its entirety; WO2006099875 (Genmab), U.S. Ser. No. 11/886,932, which is incorporated by reference in its entirety; and WO2008047242 (Sanofi-Aventis), U.S. Ser. No. 12/441,466, which is incorporated by reference in its entirety.

Combinations of antibodies specific for CD38 and other agents are described for example in WO200040265 (Research Development Foundation); WO2006099875 and WO2008037257 (Genmab); and WO2010061360, WO2010061359, WO2010061358 and WO2010061357 (Sanofi Aventis), which are all incorporated by reference in their entireties.

Preferably, an anti-CD38 antibody for the use as described herein is an antibody specific for CD38. More preferably, an anti-CD38 antibody is an antibody or antibody fragment, such as a monoclonal antibody, specifically binding to CD38 and deleting antibody-secreting cells. Such an antibody may be of any type, such as a murine, a rat, a chimeric, a humanized or a human antibody.

A “human antibody” or “human antibody fragment”, as used herein, is an antibody or antibody fragment having variable regions in which the framework and CDR regions are from sequences of human origin. If the antibody contains a constant region, the constant region also is from such sequences. Human origin includes, but is not limited to human germline sequences, or mutated versions of human germline sequences or antibody containing consensus framework sequences derived from human framework sequences analysis, for example, as described in Knappik et al., (2000) J Mol Biol 296:57-86). Human antibodies can be isolated e.g. from synthetic libraries or from transgenic mice (e.g. Xenomouse). An antibody or antibody fragment is human if its sequence is human, irrespective of the species from which the antibody is physically derived, isolated, or manufactured.

The structures and locations of immunoglobulin variable domains, e.g., CDRs, may be defined using well known numbering schemes, e.g., the Kabat numbering scheme, the Chothia numbering scheme, or a combination of Kabat and Chothia (see, e.g. Sequences of Proteins of Immunological Interest, U.S. Department of Health and Human Services (1991), eds. Kabat et al.; Lazikani et al., (1997) J. Mol. Bio. 273:927-948); Kabat et al., (1991) Sequences of Proteins of Immunological Interest, 5th edit., NIH Publication no. 91-3242 U.S. Department of Health and Human Services; Chothia et al., (1987) J. Mol. Biol. 196:901-917; Chothia et al., (1989) Nature 342:877-883; and Al-Lazikani et al., (1997) J. Mol. Biol. 273:927-948.

A “humanized antibody” or “humanized antibody fragment” is defined herein as an antibody molecule, which has constant antibody regions derived from sequences of human origin and the variable antibody regions or parts thereof or only the CDRs are derived from another species. For example, a humanized antibody can be CDR-grafted, wherein the CDRs of the variable domain are from a non-human origin, while one or more frameworks of the variable domain are of human origin and the constant domain (if any) is of human origin.

The term “chimeric antibody” or “chimeric antibody fragment” is defined herein as an antibody molecule, which has constant antibody regions derived from, or corresponding to, sequences found in one species and variable antibody regions derived from another species. Preferably, the constant antibody regions are derived from, or corresponding to, sequences found in humans, and the variable antibody regions (e.g. VH, VL, CDR or FR regions) are derived from sequences found in a non-human animal, e.g. a mouse, rat, rabbit or hamster.

The term “isolated antibody” refers to an antibody or antibody fragment that is substantially free of other antibodies or antibody fragments having different antigenic specificities. Moreover, an isolated antibody or antibody fragment may be substantially free of other cellular material and/or chemicals. Thus, in some aspects, antibodies provided are isolated antibodies, which have been separated from antibodies with a different specificity. An isolated antibody may be a monoclonal antibody. An isolated antibody may be a recombinant monoclonal antibody. An isolated antibody that specifically binds to an epitope, isoform or variant of a target may, however, have cross-reactivity to other related antigens, e.g., from other species (e.g., species homologs).

The term “monoclonal antibody” as used herein refers to a preparation of antibody molecules of single molecular composition. A monoclonal antibody composition displays a unique binding site having a unique binding specificity and affinity for particular epitopes.

In addition, as used herein, an “immunoglobulin” (Ig) hereby is defined as a protein belonging to the class IgG, IgM, IgE, IgA, or IgD (or any subclass thereof), and includes all conventionally known antibodies and functional fragments thereof. A preferred class of immunoglobulins for use in the present invention is IgG.

The phrase “antibody fragment”, as used herein, refers to one or more portions of an antibody that retain the ability to specifically interact with (e.g., by binding, steric hindrance, stabilizing spatial distribution) an antigen. Examples of binding fragments include, but are not limited to, a Fab fragment, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., (1989) Nature 341:544-548), which consists of a VH domain; and an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as “single chain Fragment (scFv)”; see e.g., Bird et al., (1988) Science 242:423-426; and Huston et al., (1988) Proc. Natl. Acad. Sci. 85:5879-5883). Such single chain antibodies are also intended to be encompassed within the term “antibody fragment”. These antibody fragments are obtained using conventional techniques known to those of skill in the art, and the fragments are screened for utility in the same manner as are intact antibodies. Antibody fragments can also be incorporated into single domain antibodies, maxibodies, minibodies, intrabodies, diabodies, triabodies, tetrabodies, v-NAR and bis-scFv (see, e.g., Hollinger and Hudson, (2005) Nature Biotechnology 23:1126-1136). Antibody fragments can be grafted into scaffolds based on polypeptides such as Fibronectin type III (Fn3) (see U.S. Pat. No. 6,703,199, which describes fibronectin polypeptide monobodies). Antibody fragments can be incorporated into single chain molecules comprising a pair of tandem Fv segments (VH-CH1-VH-CH1) which, together with complementary light chain polypeptides, form a pair of antigen-binding sites (Zapata et al., (1995) Protein Eng. 8:1057-1062; and U.S. Pat. No. 5,641,870).

The present disclosure provides therapeutic methods comprising the administration of a therapeutically effective amount of an anti-CD38 antibody as disclosed to a subject in need of such treatment. A “therapeutically effective amount” or “effective amount”, as used herein, refers to the amount of an antibody specific for CD38, necessary to elicit the desired biological response. In accordance with the subject disclosure, the therapeutic effective amount is the amount of an antibody specific for CD38 necessary to treat and/or prevent autoantibody-mediated autoimmune diseases and symptoms associated with said AD. An effective amount for a particular individual may vary, depending on factors such as the condition being treated, the overall health of the patient, the method route and dose of administration and the severity of side effects (Maynard, et al. (1996) A Handbook of SOPs for Good Clinical Practice, Interpharm Press, Boca Raton, Fla.; Dent (2001) Good Laboratory and Good Clinical Practice, London, UK).

As used herein, the terms “treat”, “treating”, or the like, mean to alleviate symptoms, eliminate the causation of symptoms either on a temporary or permanent basis, or to prevent or slow the appearance of symptoms of the named disorder or condition.

‘Preventing’ or ‘prevention’ refers to a reduction in risk of acquiring or developing a disease or disorder (i.e. causing at least one of the clinical symptoms of the disease not to develop in a subject that may be exposed to a disease-causing agent, or predisposed to the disease in advance of disease onset. “Prevention” refers to methods which aim to prevent the onset of a disease or its symptoms or which delay the onset of a disease or its symptoms.

The term ‘prophylaxis’ is related to ‘prevention’, and refers to a measure or procedure the purpose of which is to prevent, rather than to treat or cure a disease. Non-limiting examples of prophylactic measures may include the administration of vaccines; the administration of low molecular weight heparin to hospital patients at risk for thrombosis due, for example, to immobilization; the administration of an anti-malarial agent such as chloroquine, in advance of a visit to a geographical region where malaria is endemic or the risk of contracting malaria is high.

“Palliating” one or more symptoms of autoantibody-mediated AD means lessening the extent of one or more undesirable clinical manifestations in an individual or population of individuals with autoantibody-mediated AD.

“Administered” or “administration” includes but is not limited to delivery of a drug by an injectable form, such as, for example, an intravenous, intramuscular, intradermal or subcutaneous route or mucosal route, for example, as a nasal spray or aerosol for inhalation or as an ingestible solution, capsule or tablet. Preferably, the administration is by an injectable form.

As used herein, the terms “subject”, “a subject in need thereof” or the like, mean a human or a non-human animal that exhibits one or more symptoms or indicia of autoantibody-mediated autoimmune disease, and/or who has been diagnosed with autoantibody-mediated autoimmune disease. Preferably, the subject is a primate, most preferably a human patient who has been diagnosed with autoantibody-mediated autoimmune disease.

“Subject” or “species”, as used in this context refers to any mammal, including rodents, such as mouse or rat, and primates, such as cynomolgus monkey (Macaca fascicularis), rhesus monkey (Macaca mulatta) or humans (Homo sapiens). Preferably, the subject is a primate, most preferably a human.

As used herein, the term “autoantibody-mediated autoimmune diseases” includes “autoantibody-associated autoimmune diseases”, and refers to a group of diseases that are characterized by the presence of autoantibodies (autoantibody positive), in which either (i) a causative correlation and direct contribution of the autoantibodies to the pathogenesis of the disease and its associated symptoms is given or (ii) a causative correlation and direct contribution of the autoantibodies to the pathogenesis of the disease and its associated symptoms is less clear but might be given. Autoantibody-mediated autoimmune diseases include, but are not limited to the diseases exemplary listed in Table 1.

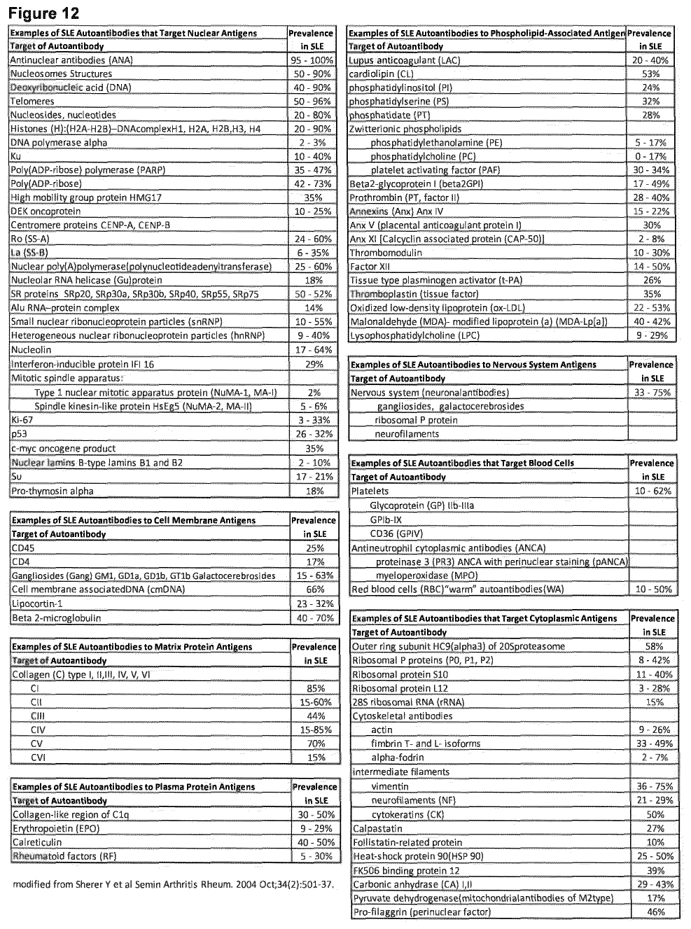

TABLE 1
Examples of autoantibody-mediated autoimmune diseases

| Autoimmune Disease | Autoantibodies (examples) | Main target organ(s) |
|---|---|---|
| Addison's disease | anti-steroidogenic cytochrome | adrenal gland |
| | P450 enzyme 21-hydroxylase | |
| ANCA-associated vasculitis | anti-neutrophil cytoplasmic abs | vasculature |
| | anti-myeloperoxidase (MPO) | |
| | anti-proteinase 3 (PR3) | |
| Antiphospholipid syndrome (APS) | anti-cardiolipin | vasculature |
| | anti-beta-2-glycoprotein | |
| Autoimmune gastritis | anti-H+/K+ ATPase | stomach |
| | anti-intrinsic factor | |
| Autoimmune haemolytic anaemia | anti-erythrocyte | red blood cells |
| Autoimmune hepatitis | anti-ASMA, | liver |
| | anti-actin, | |
| | anti-cytochrom P450 | |
| | ANA | |
| Autoimmune myopathies | anti-SRP, | skeletal muscle |
| | anti-HMGCR, | |
| | anti-myosin | |
| Autoimmune orchitis | anti-sperm antibodies | testis |
| Autoimmune pancreatitis | anti-amylase alpha2 | pancreas |
| Autoimmune thyroiditis | anti-thyroglobulin | thyroid gland |
| | anti-thyroid peroxidase | |
| Autoimmune Bullous Skin Diseases | anti-hemidesmosome, | skin, |
| (e.g. Bullous pemphigoid) | anti-dystonin, | mucous membranes |
| | anti-type XVII collagen | |
| Celiac disease | anti-transglutaminase | small intestine |
| Chronic immune polyneuropathy | anti-paranodal proteins | peripheral nervous |
| | anti-neurofascin-155, | system |
| | anti-contactin-1 | |
| | anti-caspr-1 | |
| Dermatomyositis-polymyositis | anti-muscle antigens | skeletal muscle, |
| | anti-aminoacyl tRNA synthetases | skin, lungs, heart, |
| | anti-nuclear antibodies | joints |
| Epidermolysis bullosa acquisita | anti-collagen type VII | skin |
| Keratoconjunctivitis sicca (or dry eye | anti-kallikrein 13 | ocular surface tissues |
| syndrome) | | |
| Goodpasture's disease | anti-type IV collagen, | lung, kidney |
| | anti-COL4 | |
| Graves' disease | anti-thyroid-stimulating hormone | thyroid |
| | receptor | |
| Guillian-Barré syndrome | anti-GD3, | peripheral nervous |
| | anti-ganglioside abs | system |
| Hashimoto's disease | anti-thyroid peroxidase antibodies | thyroid gland |
| | (anti-TPO) | |
| idiopathic interstitial pneumonias | several | lung |
| Idiopathic membranous | anti-PLA2R, | kidney |
| glomerulonephritis (IMN) (or primary | anti-THSD7A | |
| membranous nephropathy) | | |
| Idiopathic thrombocytopaenia (ITP) | anti-platelet glycoproteins, | platelets |
| | anti-glycoprotein IIb/IIIa | |
| | anti-glycoprotein Ib/IX | |
| | anti-glycoprotein Ia/IIa | |
| Multiple sclerosis | anti-KIR4 | central nervous |
| | anti-myelin basic protein (MBP) | system |
| | anti-proteolipid protein | (brain, spinal cord) |
| Myasthenia gravis | anti-acetylcholine receptor | skeletal muscle |
| | (anti-nicotinic AChRs) | |
| | anti-muscle specific kinase | |
| | (anti-MuSK) | |
| | anti-LRP4 | |
| Neuromyelitis optica (Devic-Syndrom) | anti-aquaporin 4 (AQP4) | central nervous |
| | | system (CNS) |
| Ovarian insufficiency | anti-HSP90, anti-HSPA5 | ovaries |
| Pemphigus foliaceus | anti-desmogleins, anti-Dsg1 | skin, |
| | | mucous membranes |
| Pemphigus vulgaris | anti-desmogleins, anti-Dsg3 | skin, |
| | | mucous membranes |
| Pernicious anemia | anti-parietal cell abs | stomach |
| Primary biliary cholangitis (PBC) | anti-mitochondrial antibodies | Small bile ducts |
| | | liver |
| Primary biliary cirrhosis | anti-2-oxoacid dehydrogenase | liver |
| Rheumatoid arthritis | anti-IgG | systemic |
| | anti-filaggrin | joints, lungs, heart etc. |
| | anti-fibrin | |
| Sjogren's syndrome | anti-Ro, anti-La, ANA | salivary gland |
| Systemic Lupus Erythematosus (SLE) | several, (see FIG 12) e.g. | systemic |
| | anti-nuclear abs (ANA), | skin, joints, kidneys, |
| | anti-dsDNA, anti-Ro, anti-Sm | brain, lungs, heart etc |
| | anti-histones, anti-nucleosomes | |
| | anti-phospholipid, anti-cardiolipin | |
| Systemic sclerosis | anti-topoisomerase 1 (ATA), | connective tissue |
| | anti-centromere (CENP), | |
| | anti-RNA polymerase III | |
| Type I diabetes | anti-insulin, | pancreatic islet cells |
| | anti-glutamic acid decarboxylase, | |
| | anti-protein tyrosine phosphatase | |
| Vitiligo | anti-tyrosinase | melanocytes |
| | anti-tyrosinase-related protein-2 | |
| Autoimmune encephalitis | anti-N-methyl-D-aspartate- | CNS |
| | receptor (anti-NMDAR) | |

As used herein, the term “about” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

“Pharmacokinetics” or “PK” as used herein describes how the body affects a specific drug after administration through the mechanisms like absorption and distribution, as well as the metabolic changes of the drug in the body, and the effects and routes of excretion of the metabolites of the drug. Pharmacokinetic properties of drugs may be affected by the route of administration and the dose of administered drug.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable vehicle” refers to a diluent, adjuvant, excipient or carrier with which an antibody or antibody fragment is administered.

Throughout this specification, unless the context requires otherwise, the words “comprise”, “have” and “include” and their respective variations such as “comprises”, “comprising”, “has”, “having”, “includes” and “including” will be understood to imply the inclusion of a stated element or integer or group of elements or integers but not the exclusion of any other element or integer or group of elements or integers.

“MOR202” is an anti-CD38 antibody, also known as “MOR03087” or “MOR3087”. The terms are used interchangeable in the present disclosure. MOR202 has an IgG1 Fc region.

The amino acid sequence of the MOR202 HCDR1 according to Kabat is:

(SEQ ID NO: 1)
SYYMN

The amino acid sequence of the MOR202 HCDR2 according to Kabat is:

(SEQ ID NO: 2)
GISGDPSNTYYADSVKG

The amino acid sequence of the MOR202 HCDR3 according to Kabat is:

(SEQ ID NO: 3)
DLPLVYTGFAY

The amino acid sequence of the MOR202 LCDR1 according to Kabat is:

(SEQ ID NO: 4)
SGDNLRHYYVY

The amino acid sequence of the MOR202 LCDR2 according to Kabat is:

(SEQ ID NO: 5)
GDSKRPS

The amino acid sequence of the MOR202 LCDR3 is: QTYTGGASL (SEQ ID NO: 6)

The amino acid sequence of the MOR202 Variable Heavy Domain is:

(SEQ ID NO: 7)
QVQLVESGGGLVQPGGSLRLSCAASGFTFSSYYMNWVRQAP
GKGLEWVSGISGDPSNTYYADSVKGRFTISRDNSKNTLYLQ
MNSLRAEDTAVYYCARDLPLVYTGFAYWGQGTLVTVSS

The amino acid sequence of the MOR202 Variable Light Domain is:

(SEQ ID NO: 8)
DIELTQPPSVSVAPGQTARISCSGDNLRHYYVYWYQQKPGQ
APVLVIYGDSKRPSGIPERFSGSNSGNTATLTISGTQAEDE
ADYYCQTYTGGASLVFGGGTKLTVLGQ

The DNA sequence encoding the MOR202 Variable Heavy Domain is:

(SEQ ID NO: 10)
CAGGTGCAATTGGTGGAAAGCGGCGGCGGCCTGGTGCAAC
CGGGCGGCAGCCTGCGTCTGAGCTGCGCGGCCTCCGGATT
TACCTTTTCTTCTTATTATATGAATTGGGTGCGCCAAGCC
CCTGGGAAGGGTCTCGAGTGGGTGAGCGGTATCTCTGGTG
ATCCTAGCAATACCTATTATGCGGATAGCGTGAAAGGCCG
TTTTACCATTTCACGTGATAATTCGAAAAACACCCTGTAT
CTGCAAATGAACAGCCTGCGTGCGGAAGATACGGCCGTGT
ATTATTGCGCGCGTGATCTTCCTCTTGTTTATACTGGTTT
TGCTTATTGGGGCCAAGGCACCCTGGTGACGGTTAGCTCA.

The DNA sequence encoding the MOR202 Variable Light Domain is:

(SEQ ID NO: 11)
GATATCGAACTGACCCAGCCGCCTTCAGTGAGCGTTGCACC
AGGTCAGACCGCGCGTATCTCGTGTAGCGGCGATAATCTTC
GTCATTATTATGTTTATTGGTACCAGCAGAAACCCGGGCAG
GCGCCAGTTCTTGTGATTTATGGTGATTCTAAGCGTCCCTC
AGGCATCCCGGAACGCTTTAGCGGATCCAACAGCGGCAACA
CCGCGACCCTGACCATTAGCGGCACTCAGGCGGAAGACGAA
GCGGATTATTATTGCCAGACTTATACTGGTGGTGCTTCTCT
TGTGTTTGGCGGCGGCACGAAGTTAACCGTTCTTGGCCAG.

The Invention

The present invention relates to an antibody, or antibody fragment, specific for CD38 useful in the prophylaxis and/or treatment of autoantibody-mediated autoimmune disease. In some aspects, the antibody is MOR202 and the autoantibody-mediated AD is anyone selected from Table 1. In one aspect, the antibody is MOR202 and the autoantibody-mediated AD is SLE. In a particular aspect, the antibody is MOR202 and the autoantibody-mediated AD is idiopathic membranous glomerulonephritis, preferably anti-PLA2R positive membranous glomerulonephritis.

The present invention also provides methods for the prophylaxis and/or treatment of autoantibody-mediated autoimmune disease comprising administering an antibody or antibody fragment, specific for CD38 to a subject in need thereof. In some aspects, the antibody, or antibody fragment, specific for CD38 used in said method is MOR202 and the autoantibody-mediated AD is anyone selected from Table 1. In one aspect, the antibody, or antibody fragment, specific for CD38 used in said method is MOR202 and the autoantibody-mediated AD is SLE. In a particular aspect, the antibody, or antibody fragment, specific for CD38 used in said method is MOR202 and the autoantibody-mediated AD is idiopathic membranous glomerulonephritis, preferably anti-PLA2R positive membranous glomerulonephritis.

The present invention also provides pharmaceutical compositions comprising said antibody, or antibody fragment, specific for CD38 and methods for the prophylaxis and/or treatment of autoantibody-mediated autoimmune disease by administering said antibody, or antibody fragment, specific for CD38.

Pharmaceutical Compositions

When employed as a pharmaceutical the antibody, or antibody fragment, specific for CD38 is typically administered in a pharmaceutical composition. Such compositions can be prepared in a manner well known in the pharmaceutical art and comprise the antibody, or antibody fragment, specific for CD38. Generally, the antibody, or antibody fragment, specific for CD38 is administered in an effective amount. The amount of the antibody, or antibody fragment, specific for CD38 actually administered will typically be determined by a physician, in the light of the relevant circumstances, including the condition to be treated, the chosen route of administration, the actual antibody, or antibody fragment, administered, the age, weight, and response of the individual patient, the severity of the patient's symptoms, and the like.

The compositions of the present disclosure are preferably pharmaceutical compositions comprising MOR202 and a pharmaceutically acceptable carrier, diluent or excipient, for the treatment of autoantibody-mediated autoimmune diseases.

The pharmaceutically acceptable carrier should be suitable for intravenous, intramuscular, subcutaneous, parenteral, spinal or epidermal administration (e.g., by injection or infusion). Pharmaceutically carriers enhance or stabilize the composition, or facilitate the preparation of the composition. Pharmaceutically acceptable carriers include solvents, dispersion media, coatings, antibacterial and antifungal agents, isotonic and absorption delaying agents, and the like that are physiologically compatible.

The composition should be sterile and fluid. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable compositions can be brought about by including in the composition an agent that delays absorption, for example, aluminum monostearate or gelatin.

A pharmaceutical composition of the present disclosure can be administered by a variety of routes known in the art. Selected routes of administration for antibodies or antibody fragments of the disclosure include intravenous, intramuscular, intradermal, intraperitoneal, subcutaneous, spinal or other parenteral routes of administration, for example by injection or infusion. Parenteral administration may represent modes of administration other than enteral and topical administration, usually by injection, and includes, without limitation, intravenous, intramuscular, intraarterial, intrathecal, intracapsular, intraorbital, intraocular, intracardiac, intradermal, intraperitoneal, transtracheal, subcutaneous, subcuticular, intraarticular, subcapsular, subarachnoid, intraspinal, epidural, intracerebral, intralesional, and intrasternal injection and infusion. Alternatively, a composition of the disclosure can be administered via a non-parenteral route, such as a topical, epidermal, cutaneous or mucosal route of administration, for example, intranasally, orally, vaginally, rectally, sublingually, transdermally or topically. Furthermore, the antibodies or antibody fragments can be administered as a sustained release formulation, in which case less frequent administration is required. In addition, pulmonary administration can also be employed, e.g., by use of an inhaler or nebulizer, and formulation with an aerosolizing agent.

The antibody, or antibody fragment, specific for CD38 is preferably formulated as injectable composition. In preferred aspects, the anti-CD38 antibody of the present disclosure is administered intravenously. In other aspects, the anti-CD38 antibody of the present disclosure is administered, subcutaneously, intraarticularly or intra-spinally.

Depending on the route of administration, the active compound, i.e. antibody, antibody fragment, bispecific and multispecific molecule, may be coated in a material to protect the compound from the action of acids and other natural conditions that may inactivate the compound.

Injectable compositions are typically based upon injectable sterile saline or phosphate-buffered saline or other injectable carriers known in the art. As before, the antibody, or antibody fragment, specific for CD38 in such compositions is typically a minor component, often being from about 0.05 to 10% by weight with the remainder being the injectable carrier and the like. Where necessary, the composition may also include a solubilizing agent and a local anesthetic such as lidocaine to ease pain at the site of the injection.

In one aspect, the present disclosure is directed to a composition comprising an anti-CD38 antibody for use in the treatment of autoantibody-mediated AD, said composition further comprising one or more pharmaceutically acceptable carriers and/or diluents.

An important aspect of the present disclosure is a pharmaceutical composition that is able to mediate killing of CD38-expressing antibody-secreting cells (e.g. plasmablasts, plasma cells) by ADCC and ADCP.

Methods of Treatment

In one embodiment, the present invention provides the antibody, or antibody fragment, specific for CD38, or pharmaceutical compositions comprising an antibody, or antibody fragment, specific for CD38, for use in the prophylaxis and/or treatment of autoantibody-mediated autoimmune disease.

In one embodiment, the present disclosure provides the antibody, or antibody fragment, specific for CD38, or pharmaceutical compositions comprising an antibody, or antibody fragment, specific for CD38, for use in the prophylaxis and/or treatment of systemic lupus erythematosus (SLE).

In another embodiment, the present disclosure provides the antibody, or antibody fragment, specific for CD38, or pharmaceutical compositions comprising an antibody, or antibody fragment, specific for CD38, for use in the prophylaxis and/or treatment of idiopathic membranous nephropathy.

In one embodiment, the present invention provides the antibody, or antibody fragment, specific for CD38, or pharmaceutical compositions comprising an antibody, or antibody fragment, specific for CD38, for use in the prophylaxis and/or treatment of autoimmune membranous nephropathy.

In a particular embodiment, the present disclosure provides the antibody, or antibody fragment, specific for CD38, or pharmaceutical compositions comprising an antibody, or antibody fragment, specific for CD38, for use in the prophylaxis and/or treatment of anti-PLA2R positive membranous nephropathy.

In another aspect, the present disclosure provides the antibody, or antibody fragment, specific for CD38, or pharmaceutical compositions comprising an antibody, or antibody fragment, specific for CD38, for use in the prophylaxis and/or treatment of membranous nephropathy in patients with anti-PLA2R antibody titers.

In another embodiment, the present disclosure provides the antibody, or antibody fragment, specific for CD38, or pharmaceutical compositions comprising the antibody, or antibody fragment, specific for CD38 for use in the manufacture of a medicament for use in the prophylaxis and/or treatment of autoantibody-mediated autoimmune disease.

In one aspect, the present disclosure provides the use of an anti-CD38 antibody in the preparation of a medicament for the treatment and/or prophylaxis of systemic lupus erythematosus (SLE).

In another aspect, the present disclosure provides the use of an anti-CD38 antibody in the preparation of a medicament for the treatment and/or prophylaxis of idiopathic membranous nephropathy.

In another aspect, the present disclosure provides the use of an anti-CD38 antibody in the preparation of a medicament for the treatment and/or prophylaxis of autoantibody-mediated membranous nephropathy.

In a preferred aspect, the present disclosure provides the use of an anti-CD38 antibody in the preparation of a medicament for the treatment and/or prophylaxis of anti-PLA2R positive membranous nephropathy.

In other aspects, the present disclosure provides the use of MOR202 in the preparation of a medicament in the treatment and/or prophylaxis of autoantibody-mediated autoimmune disease.

In other aspects, the present disclosure provides the use of MOR202 in the preparation of a medicament in the treatment and/or prophylaxis of systemic lupus erythematosus (SLE).

In other aspects, the present disclosure provides the use of MOR202 in the preparation of a medicament in the treatment and/or prophylaxis of idiopathic membranous nephropathy.

In other aspects, the present disclosure provides the use of MOR202 in the preparation of a medicament in the treatment and/or prophylaxis of autoantibody-mediated membranous nephropathy.

In a preferred aspect, the present disclosure provides the use of MOR202 in the preparation of a medicament for the treatment and/or prophylaxis of anti-PLA2R positive membranous nephropathy.

In one embodiment, the present disclosure provides the antibody, or antibody fragment, specific for CD38 and another therapeutic agent or pharmaceutical compositions comprising the antibody, or antibody fragment, specific for CD38 and another therapeutic agent, for use in the prophylaxis and/or treatment of autoantibody-mediated autoimmune disease, preferably autoantibody-mediated membranous nephropathy.

In another embodiment, the present disclosure provides the antibody, or antibody fragment, specific for CD38 and another therapeutic agent or pharmaceutical compositions comprising the antibody, or antibody fragment, specific for CD38 and another therapeutic agent, for use in the prophylaxis and/or treatment of systemic lupus erythematosus (SLE).

In another embodiment, the present disclosure provides the antibody, or antibody fragment, specific for CD38 and another therapeutic agent or pharmaceutical compositions comprising the antibody, or antibody fragment, specific for CD38 and another therapeutic agent, for use in the prophylaxis and/or treatment of idiopathic membranous nephropathy.

In a preferred embodiment, the present disclosure provides the antibody, or antibody fragment, specific for CD38 and another therapeutic agent or pharmaceutical compositions comprising the antibody, or antibody fragment, specific for CD38 and another therapeutic agent, for use in the prophylaxis and/or treatment of anti-PLA2R positive membranous nephropathy.

In one embodiment, the present disclosure provides the antibody, or antibody fragment, specific for CD38 and another therapeutic agent, or pharmaceutical compositions comprising the antibody, or antibody fragment, specific for CD38 and another therapeutic agent for use in the manufacture of a medicament for use in the prophylaxis and/or treatment of autoantibody-mediated autoimmune disease, preferably autoantibody-mediated membranous nephropathy.

In other aspects, the present disclosure provides the use of an anti-CD38 antibody and another therapeutic agent, or pharmaceutical compositions comprising the anti-CD38 antibody or antibody fragment, in the preparation of a medicament for the treatment and/or prophylaxis of autoantibody-mediated autoimmune disease, preferably autoantibody-mediated membranous nephropathy.

In preferred aspects, the present disclosure provides the use of an anti-CD38 antibody and another therapeutic agent, or pharmaceutical compositions comprising the anti-CD38 antibody or antibody fragment, in the preparation of a medicament for the treatment and/or prophylaxis of systemic lupus erythematosus (SLE).

In preferred aspects, the present disclosure provides the use of an anti-CD38 antibody and another therapeutic agent, or pharmaceutical compositions comprising the anti-CD38 antibody or antibody fragment, in the preparation of a medicament for the treatment and/or prophylaxis of idiopathic membranous nephropathy.

In other aspects, the present disclosure provides the use of MOR202 and another therapeutic agent, or pharmaceutical compositions comprising MOR202, in the preparation of a medicament for the treatment and/or prophylaxis of autoantibody-mediated autoimmune disease, preferably autoantibody-mediated membranous nephropathy.

In one aspect, the present disclosure provides the use of MOR202 and another therapeutic agent, or pharmaceutical compositions comprising MOR202, in the preparation of a medicament for the treatment and/or prophylaxis of systemic lupus erythematosus (SLE).

In a particular aspect, the present disclosure provides the use of MOR202 and another therapeutic agent, or pharmaceutical compositions comprising MOR202, in the preparation of a medicament for the treatment and/or prophylaxis of idiopathic membranous nephropathy.

In a particular aspect, the present disclosure provides the use of MOR202 and another therapeutic agent, or pharmaceutical compositions comprising MOR202, in the preparation of a medicament for the treatment and/or prophylaxis of anti-PLA2R positive membranous nephropathy.

In a particular embodiment, said another therapeutic agent is an autoimmune disease treatment agent. In a particular embodiment, said agent is an immunosuppressive agent and selected from the group comprising steroids (e.g. clobetasol propionate, desoximetasone, hydrocortisone, methylprednisolone, prednisone, prednisolone, budesonide, or dexamethasone), proteasome inhibitors (e.g. bortezomib), cytostatics (e.g. cyclophosphamide, azathioprine, methotrexate), drugs acting on immunophilins (e.g. ciclosporin, tacrolimus, sirolimus) and other immunosuppressants.

In additional method of treatment aspects, this invention provides methods of prophylaxis and/or treatment of a mammal afflicted with an autoantibody-mediated autoimmune disease, which methods comprise the administration of an effective amount of the antibody, or antibody fragment, specific for CD38 or one or more of the pharmaceutical compositions herein described for the treatment and/or prophylaxis of said condition.

In one aspect, the present invention provides a method for the treatment of autoantibody-mediated AD, preferably autoantibody-mediated membranous nephropathy. comprising administering to said subject an anti-CD38 antibody.

In one embodiment, the present disclosure provides methods of prophylaxis and/or treatment of a mammal afflicted with an autoantibody-mediated autoimmune disease, wherein said methods comprise an administration of another therapeutic agent with the antibody, or antibody fragment, specific for CD38. In a particular embodiment, said other therapeutic agent is an autoimmune disease treatment agent. In a particular embodiment said agent is an immunosuppressive agent.

In the method of treatment or use described herein, the autoimmune disease is particularly an autoantibody-mediated autoimmune disease (e.g. SLE, Graves' Disease, Myasthenia Gravis, pemphigus vulgaris, autoimmune encephalitis, idiopathic membranous glomerulonephritis, anti-PLA2R positive membranous glomerulonephritis).

In a particular aspect, the present disclosure provides a method for the treatment and or/prophylaxis of anti-PLA2R positive membranous glomerulonephritis in a subject, said method comprising administering an anti-CD38 antibody to said subject.

In one embodiment, the present disclosure provides methods of prophylaxis and/or treatment of a subject suffering from moderate-to-severe autoantibody-mediated AD, which methods comprise the administration of an effective amount of the antibody, or antibody fragment, specific for CD38 or one or more of the pharmaceutical compositions herein described for the treatment and/or prophylaxis of said condition.

In some embodiments, the present disclosure provides methods of prophylaxis and/or treatment of subjects suffering from autoantibody-mediated AD, wherein said subject is resistant to treatment by other immunosuppressant therapies, including corticosteroids or calcineurin inhibitors or B cell depleting therapies (e.g. with Rituximab or any other anti-CD20 antibody, or anti-BAFF antibody), which methods comprise the administration of an effective amount of the antibody, or antibody fragment, specific for CD38 or one or more of the pharmaceutical compositions herein described for the treatment and/or prophylaxis of said condition.

In one aspect, the invention provides methods of using an anti-CD38 antibody or antibody fragment to achieve a prophylactic or therapeutic benefit in patients with autoantibody-mediated autoimmune disease, preferably autoantibody-mediated membranous nephropathy.

In another aspect provided herein are methods using an anti-CD38 antibody to treat and/or prevent symptoms mediated with autoantibody-mediated autoimmune disease.

In another aspect provided herein are methods for reducing the incidence of autoantibody-mediated disease symptoms, ameliorating autoantibody-mediated disease symptoms, suppressing autoantibody-mediated disease symptoms, palliating autoantibody-mediated disease symptoms, and/or delaying the onset, development, or progression of autoantibody-mediated disease in a subject, said method comprising administering an effective amount of an anti-CD38 antibody to the subject.

In preferred embodiments, the disclosure provides methods to treat patients that show elevated levels of one or more autoantibody specificities associated with the autoimmune disease.

In other aspects, the present disclosure provides a method for the treatment and/or prevention of SLE caused by the presence of anti-nuclear or anti-DNA autoantibodies or any other SLE autoantibody as listed in FIG. 12.

In yet other aspects, the present invention provides a method for the treatment and/or prevention of SLE associated with the presence of anti-nuclear or anti-DNA autoantibodies or any other SLE autoantibody as listed in FIG. 12.

In other aspects, the present disclosure provides a method for the treatment and/or prevention of a disease caused by the presence of anti-phospholipase A2 receptor (PLA2R) autoantibodies. In yet other aspects, the present invention provides a method for the treatment and/or prevention of a disease associated with the presence of anti-phospholipase A2 receptor (PLA2R) autoantibodies.

In other aspects, the present disclosure provides a method for the treatment and/or prevention of a disease caused by the presence of anti-thrombospondin type-1 domain-containing 7A autoantibodies. In yet other aspects, the present invention provides a method for the treatment and/or prevention of a disease associated with the presence of anti-thrombospondin type-1 domain-containing 7A autoantibodies.

In other embodiments, the disclosure provides methods to reduce autoantibody titers in serum of subjects suffering from autoantibody-mediated autoimmune disease, which methods comprise the administration of an effective amount of the antibody, or antibody fragment, specific for CD38 or one or more of the pharmaceutical compositions herein described.

In a preferred embodiment, the disclosure provides methods to reduce autoantibody titers in serum of subjects suffering from idiopathic membranous glomerulonephritis, which methods comprise the administration of an effective amount of the antibody, or antibody fragment, specific for CD38 or one or more of the pharmaceutical compositions herein described. For example, the methods provided herein comprise administering an anti-CD38 antibody to patients with elevated levels of anti-PLA2R and/or anti-thrombospondin type-1 domain-containing 7A autoantibodies.

In one embodiment, the reduction (change) of autoantibody titers in serum of subjects suffering from anti-PLA2R positive membranous glomerulonephritis is at least 5%, at least 10%, at least 15%, at least 20%, at least 25%, at least 30%, at least 35%, at least 40%, at least 45%, or at least 50% compared to baseline after administering an antibody, or antibody fragment, specific for CD38 or one or more of the pharmaceutical compositions herein described.

In another embodiment, the disclosure provides methods for treating and/or prophylaxis of proteinuria associated with anti-PLA2R positive membranous glomerulonephritis in an individual, which methods comprise the administration of an effective amount of the antibody, or antibody fragment, specific for CD38 or one or more of the pharmaceutical compositions herein described.

In another aspect, the disclosure provides methods for preventing the decline of renal function in an individual with anti-PLA2R positive membranous nephropathy, which methods comprise the administration of an effective amount of the antibody, or antibody fragment, specific for CD38 or one or more of the pharmaceutical compositions herein described.

In another aspect, the disclosure provides methods for treating and/or prophylaxis of hypercholesterolemia (high cholesterol) in an individual with membranous nephropathy, which methods comprise the administration of an effective amount of the antibody, or antibody fragment, specific for CD38 or one or more of the pharmaceutical compositions herein described.

In one embodiment, the present disclosure refers to the use of an antibody or antibody fragment specific for CD38 for the treatment of autoantibody-mediated autoimmune disease, wherein said antibody or antibody fragment binds to a CD38 expressing plasma cell.

In further embodiments, the present disclosure refers to a method for the treatment of autoantibody-mediated autoimmune disease in a subject, comprising administering to the subject a pharmaceutical composition comprising an antibody or antibody fragment that binds to a CD38 expressing cell and leads to the depletion of such CD38 expressing cell.

In a preferred embodiment, the present disclosure refers to a method for the treatment of autoantibody-mediated autoimmune disease in a subject, comprising administering to the subject a pharmaceutical composition comprising an antibody or antibody fragment that binds to a CD38 expressing antibody-secreting cell and leads to the depletion of such CD38 expressing antibody-secreting cell, while sparing other (antibody-non-secreting) cells with low CD38 expression such as NK cells or the like.

In a particular preferred embodiment, the present disclosure refers to a method for the treatment of autoantibody-mediated autoimmune disease in a subject, comprising administering to the subject a pharmaceutical composition comprising an antibody or antibody fragment that binds to a CD38 expressing antibody-secreting cell and leads to the depletion of such CD38 expressing antibody-secreting cell, while sparing NK cells, i.e. wherein the antibody shows a significant higher specific cell killing on antibody-secreting cells than on NK cells.

In one embodiment, the present disclosure refers to a method for the treatment of autoantibody-mediated autoimmune disease in a subject, comprising administering to the subject a pharmaceutical composition comprising an antibody or antibody fragment that binds to a CD38 expressing antibody-secreting cell and leads to the depletion of such CD38 expressing antibody-secreting cell, while sparing other (antibody-non-secreting) cells with low CD38 expression such as NK cells or the like, wherein the specific cell killing of the antibody-secreting plasma cell is at least 10%, at least 15%, at least 20%, at least 25%, at least 30%, at least 35%, at least 40% and wherein the specific cell killing of antibody-non-secreting NK cells is less than 30%, less than 25%, less than 20%, or less than 15% as determined in a standard ADCC assay.

The antibody, or antibody fragment, specific for CD38 can be administered as the sole active agent or it can be administered in combination with other therapeutic agents. In a specific embodiment, co-administration of two (or more) agents allows for significantly lower doses of each to be used, thereby reducing the side effects seen.

In one embodiment, the antibody, or antibody fragment, specific for CD38 or a pharmaceutical composition comprising the antibody, or antibody fragment, specific for CD38 is administered as a medicament. In a specific embodiment, said pharmaceutical composition additionally comprises a further active ingredient.

By co-administration is included any means of delivering two or more therapeutic agents to the patient as part of the same treatment regimen, as will be apparent to the skilled person. Whilst the two or more agents may be administered simultaneously in a single formulation, i.e. as a single pharmaceutical composition, this is not essential. The agents may be administered in different formulations and at different times.

The therapies (e.g., prophylactic or therapeutic agents) of the combination therapies of the present disclosure can be administered concomitantly or sequentially to a subject.

The therapy (e.g., prophylactic or therapeutic agents) of the combination therapies of the present disclosure can also be cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time and repeating this sequential administration, i.e., the cycle, in order to reduce the development of resistance to one of the therapies (e.g., agents) to avoid or reduce the side effects of one of the therapies (e.g., agents), and/or to improve, the efficacy of the therapies.

The therapies (e.g., prophylactic or therapeutic agents) of the combination therapies of the disclosure can be administered to a subject concurrently. The term “concurrently” is not limited to the administration of therapies (e.g., prophylactic or therapeutic agents) at exactly the same time, but rather it is meant that a pharmaceutical composition comprising antibodies or antibody fragments of the disclosure are administered to a subject in a sequence and within a time interval such that the antibodies of the disclosure can act together with the other therapy(ies) to provide an increased benefit than if they were administered otherwise.

Antibody

In certain embodiments of the present disclosure, the antibody or antibody fragment specific for CD38 according to the present disclosure comprises a variable heavy chain variable region, a variable light chain region, heavy chain, light chain and/or CDRs comprising any of the amino acid sequences of the CD38 specific antibodies as set forth in WO2007/042309.

In an embodiment, said antibody or antibody fragment specific for CD38 comprises a HCDR1 region comprising the amino acid sequence of SEQ ID NO: 1, a HCDR2 region comprising the amino acid sequence of SEQ ID NO: 2, a HCDR3 region comprising the amino acid sequence of SEQ ID NO: 3, a LCDR1 region comprising the amino acid sequence of SEQ ID NO: 4, a LCDR2 region comprising the amino acid sequence of SEQ ID NO: 5 and a LCDR3 region comprising the amino acid sequence of SEQ ID NO: 6.

In one embodiment, said antibody or antibody fragment specific for CD38, comprises the HCDR1 region of SEQ ID NO: 1, the HCDR2 region of SEQ ID NO: 2, the HCDR3 region of SEQ ID NO: 3, the LCDR1 region of SEQ ID NO: 4, the LCDR2 region of SEQ ID NO: 5 and the LCDR3 region of SEQ ID NO: 6.

In an embodiment, said antibody or antibody fragment specific for CD38 comprises a variable heavy chain region of SEQ ID NO: 7 and a variable light chain region of SEQ ID NO: 8.

In another embodiment said antibody or antibody fragment comprises a variable heavy chain region of SEQ ID NO: 7 and a variable light chain region of SEQ ID NO: 8 or a variable heavy chain region and a variable light chain region that has at least 60%, at least 70%, at least 80%, at least 90% or at least 95% identity to the a variable heavy chain region of SEQ ID NO: 7 and to the variable light chain region of SEQ ID NO: 8.

An exemplary antibody or antibody fragment comprising the variable heavy chain region comprising the amino acid sequence of SEQ ID NO: 7 and a variable light chain region comprising the amino acid sequence of SEQ ID NO: 8 is the human anti-CD38 antibody known as MOR202.

In one embodiment, the present disclosure refers to a nucleic acid composition comprising a nucleic acid sequence or a plurality of nucleic acid sequences encoding said antibody or antibody fragment specific for CD38, wherein said antibody or antibody fragment comprises the HCDR1 region of SEQ ID NO: 1, the HCDR2 region of SEQ ID NO: 2, the HCDR3 region of SEQ ID NO: 3, the LCDR1 region of SEQ ID NO: 4, the LCDR2 region of SEQ ID NO: 5 and the LCDR3 region of SEQ ID NO: 6.

In another embodiment, the disclosure refers to a nucleic acid encoding an isolated monoclonal antibody or fragment thereof wherein the nucleic acid comprises a VH of SEQ ID NO: 10 and a VL of SEQ ID NO: 11.

In one embodiment, the disclosed antibody or antibody fragment specific for CD38 is a monoclonal antibody or antibody fragment.

In one embodiment, the disclosed antibody or antibody fragment specific for CD38 is a human, humanized or chimeric antibody.

In certain embodiments, said antibody or antibody fragment specific for CD38 is an isolated antibody or antibody fragment.

In another embodiment, said antibody or antibody fragment is a recombinant antibody or antibody fragment.

In a further embodiment, said antibody or antibody fragment is a recombinant human antibody or antibody fragment.

In a further embodiment, said recombinant human antibody or antibody fragment is an isolated recombinant human antibody or antibody fragment.

In a further embodiment, said recombinant human antibody or antibody fragment or isolated recombinant human antibody or antibody fragment is monoclonal.

In one embodiment, the disclosed antibody or antibody fragment is of the IgG isotype.

In another embodiment, said antibody is an IgG1.

In one embodiment said antibody fragment is a bivalent antibody fragment.

In particular aspects of the present invention, the anti-CD38 antibody is MOR202.

In an embodiment, the present disclosure refers to a pharmaceutical composition comprising MOR202 or fragment thereof specific for CD38 and a pharmaceutically acceptable carrier or excipient.

In certain embodiments, the antibody or antibody fragment specific for CD38 is an antibody or antibody fragment that specifically binds CD38.

In certain embodiments, said antibody or antibody fragment specific for CD38 is an antibody or antibody fragment that specifically binds to human CD38.

In certain embodiments, said antibody or antibody fragment specific for CD38 is an isolated monoclonal antibody or antibody fragment that specifically binds to human CD38.

In another embodiment, the present disclosure provides an antibody or antibody fragment specific for CD38, which depletes CD38 expressing antibody-secreting cells.

In a preferred aspect, the disclosure provides a preventive and/or therapeutic agent for reducing serum autoantibody levels in subjects with SLE, said agent comprising an anti-CD38 antibody as an active ingredient

In a preferred aspect, the disclosure provides a preventive and/or therapeutic agent for reducing serum autoantibody levels in subjects with aMN, said agent comprising an anti-CD38 antibody as an active ingredient.

In a particular aspect, the disclosure provides a preventive and/or therapeutic agent for reducing serum anti-PLA2R autoantibody levels in subjects with aMN, said agent comprising an anti-CD38 antibody as an active ingredient.

In another aspect, the disclosure provides a preventive and/or therapeutic agent for reducing anti-PLA2R autoantibodies deposited in kidneys of subjects with aMN, said agent comprising an anti-CD38 antibody as an active ingredient.

In a further aspect, the disclosure provides a preventive and/or therapeutic agent for reducing proteinuria in subjects with aMN, said agent comprising an anti-CD38 antibody as an active ingredient.

In another aspect, the disclosure provides a preventive and/or therapeutic agent for reducing hyperlipidemia (e.g. hypercholesterinemia, high cholesterol) in subjects with aMN, said agent comprising an anti-CD38 antibody as an active ingredient.

In another aspect, the disclosure provides a preventive and/or therapeutic agent for restoring, ameliorating or normalizing kidney function indicated by glomerular filtration rate (eGFR) based on the CKD-epi equation in subjects with aMN, said agent comprising an anti-CD38 antibody as an active ingredient.

### Working Examples

The exemplary antibody specific for CD38 used in the following examples is the human antibody MOR202.

### Example 1: Effectiveness of MOR202 on Preexisting Antibody Titers to Tetanus Toxoid as Vaccine Antigen

To assess the impact of MOR202 treatment on preexisting antibody titers, the present inventors determined anti-tetanus toxoid titers in human serum collected from subjects at defined time points after MOR202 administration.

### 1.1. Study Design

The following bioanalytical assessment is part of an open-label, multicenter, dose-escalation clinical study to characterize the safety and preliminary efficacy of the human anti-CD38 antibody MOR03087 in adult subjects with relapsed/refractory multiple myeloma. Purpose of the present experiment was the quantitative determination of anti-tetanus toxoid (anti-TT) IgG antibody titers in human serum samples obtained during the study to demonstrate that a monoclonal anti-CD38 antibody (MOR03087=MOR202) is effective in decreasing pre-existing antibody titers. Human serum samples were analyzed for anti-tetanus toxoid (anti-TT) IgG levels by ELISA (Table 4).

### 1.2. Determination of Anti-Tetanus Toxoid IaG by Quantitative ELISA

Serum samples were stored at −75±15° C. until analysis. For the determination of anti-tetanus toxoid IgG in the samples a commercially available immunoassay kit (VaccZyme™, Binding Site, product code MK010) was used. The assay was qualified at the bioanalytical test site before sample analysis and all measurements were performed in accordance with the manufacturer's recommendations. Two quality control (QC) samples with batch specific target values and ranges were provided with the kit. QC target values (high QC/low QC): 1.31/0.22 IU/mL (batch 1), 1.3210.23 IU/mL (batch 2), 1.39/0.25 IU/mL (batch 3), 1.3/0.25 IU/mL (batch 4), 1.27/0.28 IU/mL (batch 5). During the qualifying runs, 3 additional concentration levels were evaluated according to the results of the qualifying runs: ULOQ (7 IU/mL), LLOQ (0.01 IU/mL), HQC (2.8-3.5 IU/mL) (ULOQ: upper limit of quantification, LLOQ: lower limit of quantification, HQC: high quality control). The calibration standard samples were provided with the kit ready to use. One set of calibration standards consisted of: 0.01, 0.03, 0.09, 0.26, 0.78, 2.33, 7 IU/mL.

### 1.2.1. Performance of Measurement

Samples were analyzed as duplicates in runs (one run=one 96-well plate) together with one set of calibration standard samples and two sets of QC samples as provided in the assay kit. No sample work up is necessary for the performance of anti-TT IgG ELISA. Samples were measured after dilution (minimum required dilution 1:101) with sample diluent.

### 1.2.2. Principle of Test

The VaccZyme™ Anti-Tetanus Toxoid IgG Enzyme Immunoassay Kit is a two-step enzyme-linked immunosorbent assay. Wells of 12 break apart 8 well strips are coated with tetanus toxoid from Clostridium tetani. The calibrators, controls, and diluted serum samples are added to the wells and antibodies recognizing the tetanus toxoid antigen bind during the first incubation. After washing the wells to remove all unbound proteins, purified peroxidase labelled rabbit anti-human IgG (gamma-chain specific) conjugate is added. The conjugate binds to the captured human antibody and the excess unbound conjugate is removed by a further wash step. The bound conjugate is visualized with 3,3′,5,5′ tetramethylbenzidine (TMB) substrate which gives a blue reaction product, the intensity of which is proportional to the concentration of antibody in the sample. Phosphoric acid is added to each well to stop the reaction. This produces a yellow end point color, which is read at 450 nm.

### 1.2.3. Data Evaluation

Data reduction of the output from the microplate reader was performed using the Magellan™ Software version 6.6 from the TECAN Austria GmbH, using the 4-parameter logistic. The optical density of the quality control and study samples was converted into concentrations (IU/mL) using the standard curve. An extrapolation was performed (extrapolation factor 1.1) to be able to compute concentrations close to the upper and lower limit of quantification. All measured and calculated concentration data are reported with 3 significant digits.

### 1.2.4. Results

Human serum samples were analyzed in 22 assay runs. The inter-assay accuracy and precision data were evaluated from calibration standard samples in 22 accepted runs. The accuracy (expressed as bias) and the precision (expressed as coefficient of variation; CV) data are shown in Table 2.

TABLE 2
Accuracy and Precision for Calibration Standards

| | | STD | STD | STD | STD | STD | STD | STD |
|---|---|---|---|---|---|---|---|---|
| | | 0.0100 | 0.0300 | 0.0900 | 0.260 | 0.780 | 2.33 | 7.00 |
| | | IU/mL | IU/mL | IU/mL | IU/mL | IU/mL | IU/mL | IU/mL |
| Target | (IU/mL) | 0.0100 | 0.0300 | 0.0900 | 0.260 | 0.780 | 2.33 | 7.00 |
| Count | | 21 ^{a} | 22 | 22 | 22 | 22 | 22 | 22 |
| Mean | (IU/mL) | 0.00922 | 0.0295 | 0.0904 | 0.265 | 0.776 | 2.33 | 7.01 |
| SD | (IU/mL) | 0.00338 | 0.00177 | 0.00438 | 0.00726 | 0.0117 | 0.0130 | 0.0146 |
| CV | (%) | 36.6 | 6.01 | 4.84 | 2.74 | 1.51 | 0.556 | 0.208 |
| Bias | (%) | −7.76 | −1.55 | 0.449 | 1.81 | −0.456 | 0.0581 | 0.132 |

The inter-assay accuracy and precision data were evaluated from a maximum of 22 sets of QC samples in 22 accepted runs. The accuracy (expressed as bias) and the precision (expressed as coefficient of variation; CV) data are shown in Table 3.

TABLE 3
Accuracy and Precision for Quality Control Samples

| | | QC | QC | QC | QC | QC | QC | QC | QC | QC |
|---|---|---|---|---|---|---|---|---|---|---|
| | | 0.220 | 0.230 | 0.250 | 0.280 | 1.27 | 1.30 | 1.31 | 1.32 | 1.39 |
| Target | (IU/mL) | 0.220 | 0.230 | 0.250 | 0.280 | 1.27 | 1.30 | 1.31 | 1.32 | 1.39 |
| Count | | | 6 | 12 | 22 | 22 | 8 | 4 | 6 | 4 |
| Mean | (IU/mL) | 0.246 | 0.244 | 0.259 | 0.266 | 1.17 | 1.32 | 1.32 | 1.35 | 1.43 |
| SD | (IU/mL) | 0.00428 | 0.00600 | 0.00816 | 0.0150 | 0.0832 | 0.0581 | 0.0143 | 0.0673 | 0.0331 |
| CV | (%) | 1.74 | 2.46 | 3.16 | 5.66 | 7.09 | 4.42 | 1.08 | 5.00 | 2.31 |
| Bias | (%) | 11.8 | 5.92 | 3.48 | −5.17 | −7.63 | 1.22 | 0.931 | 1.93 | 3.01 |

The anti-0 concentration (IU/mL) of serum samples from 74 subjects for which baseline and at least one of “cycle 1, day 15” or “cycle 2, day 15” data points were available are shown in Table 4. Subjects that received co-medication during the clinical study (such as MG administration or boost vaccination) were not included in the analysis as these co-medication factors lead to biased results.

TABLE 4
Anti-TT antibody concentration in human serum samples after MOR202
administration

| | | final | | | |
|---|---|---|---|---|---|
| | | conc. | | change | |
| subject | nominal | [IU/mL] | % CV | [%] | FIG. |
| 10002 | baseline | 0.045 | 4.9 | | |
| 10002 | Cycle 1, Day 15 | 0.028 | 1.4 | −37.8% | 5 |
| 10002 | Cycle 2, Day 15 | 0.035 | 0.0 | −22.2% | 6 |
| 10004 | baseline | 0.977 | 8.1 | | |
| 10004 | Cycle 1, Day 15 | 0.848 | 6.2 | −13.2% | 5 |
| 10004 | Cycle 2, Day 15 | 0.854 | 5.8 | −12.6% | 6 |
| 10005 | baseline | 0.029 | 0.0 | | |
| 10005 | Cycle 1, Day 15 | 0.034 | 0.0 | 17.2% | 5 |
| 11001 | baseline | 0.439 | 3.4 | | |
| 11001 | Cycle 1, Day 15 | 0.304 | 2.2 | −30.8% | 5 |
| 11001 | Cycle 2, Day 15 | 0.332 | 0.3 | −24.4% | 6 |
| 11002 | baseline | 0.171 | 5.0 | | |
| 11002 | Cycle 1, Day 15 | 0.176 | 0.7 | 2.9% | 5 |
| 11003 | baseline | 1.200 | 3.0 | | |
| 11003 | Cycle 1, Day 15 | 0.922 | 10.9 | −23.2% | 5 |
| 11004 | baseline | 0.114 | 1.2 | | |
| 11004 | Cycle 1, Day 15 | 0.115 | 0.0 | 0.9% | |
| 11006 | baseline | 0.965 | 6.9 | | |
| 11006 | Cycle 1, Day 15 | 0.764 | 6.9 | −20.8% | 5 |
| 11006 | Cycle 2, Day 15 | 0.572 | 6.4 | −40.7% | 6 |
| 11008 | baseline | 0.346 | 0.1 | | |
| 11008 | Cycle 1, Day 15 | 0.207 | 0.9 | −40.2% | 5 |
| 11008 | Cycle 2, Day 15 | 0.097 | 6.0 | −72.0% | 6 |
| 11010 | baseline | 0.192 | 0.8 | | |
| 11010 | Cycle 1, Day 15 | 0.091 | 1.4 | −52.6% | 5 |
| 11010 | Cycle 2, Day 15 | 0.035 | 1.2 | −81.8% | 6 |
| 11011 | baseline | 0.548 | 0.8 | | |
| 11011 | Cycle 1, Day 15 | 0.211 | 2.6 | −61.5% | 5 |
| 11011 | Cycle 2, Day 15 | 0.165 | 4.1 | −69.9% | 6 |
| 11012 | baseline | 0.258 | 2.1 | | |
| 11012 | Cycle 1, Day 15 | 0.060 | 6.2 | −76.7% | 5 |
| 11013 | baseline | 0.091 | 21.7 | | |
| 11013 | Cycle 2, Day 15 | 0.070 | 14.6 | −23.1% | 6 |
| 11016 | baseline | 0.088 | 16.1 | | |
| 11016 | Cycle 1, Day 15 | 0.055 | 8.7 | −37.5% | 5 |
| 11016 | Cycle 2, Day 15 | 0.047 | 2.1 | −46.6% | 6 |
| 11017 | baseline | 0.217 | 3.1 | | |
| 11017 | Cycle 1, Day 15 | 0.154 | 6.0 | −29.0% | 5 |
| 11017 | Cycle 2, Day 15 | 0.123 | 7.6 | −43.3% | 6 |
| 11018 | baseline | 2.960 | 1.7 | | |
| 11018 | Cycle 1, Day 15 | 4.680 | 0.2 | 58.1% | 5 |
| 11018 | Cycle 2, Day 15 | 3.940 | 2.2 | 33.1% | 6 |
| 12001 | baseline | 3.490 | 11.0 | | |
| 12001 | Cycle 1, Day 15 | 1.630 | 3.8 | −53.3% | 5 |
| 12002 | baseline | 0.082 | 1.7 | | |
| 12002 | Cycle 1, Day 15 | 0.055 | 0.0 | −32.9% | 5 |
| 12002 | Cycle 2, Day 15 | 0.062 | 4.3 | −24.4% | 6 |
| 12007 | baseline | 0.936 | 4.2 | | |
| 12007 | Cycle 1, Day 15 | 0.873 | 0.5 | −6.7% | 5 |
| 12007 | Cycle 2, Day 15 | 0.638 | 3.1 | −31.8% | 6 |
| 12008 | baseline | 0.491 | 1.4 | | |
| 12008 | Cycle 1, Day 15 | 0.348 | 2.8 | −29.1% | 5 |
| 12011 | baseline | 0.019 | 24.1 | | |
| 12011 | Cycle 1, Day 15 | 0.012 | 4.6 | −36.8% | 5 |
| 12011 | Cycle 2, Day 15 | 0.009 | 24.6 | −52.6% | 6 |
| 12012 | baseline | 0.080 | 1.2 | | |
| 12012 | Cycle 1, Day 15 | 0.071 | 0.7 | −11.3% | 5 |
| 12012 | Cycle 2, Day 15 | 0.065 | 0.0 | −18.8% | 6 |
| 12013 | baseline | 0.217 | 1.6 | | |
| 12013 | Cycle 1, Day 15 | 0.140 | 1.7 | −35.5% | 5 |
| 12013 | Cycle 2, Day 15 | 0.135 | 5.2 | −37.8% | 6 |
| 12014 | baseline | 0.094 | 0.0 | | |
| 12014 | Cycle 1, Day 15 | 0.097 | 0.5 | 3.2% | 5 |
| 12015 | baseline | 0.125 | 1.3 | | |
| 12015 | Cycle 1, Day 15 | 0.129 | 2.5 | 3.2% | 5 |
| 12015 | Cycle 2, Day 15 | 0.080 | 5.5 | −36.0% | 6 |
| 12016 | baseline | 0.117 | 0.6 | | |
| 12016 | Cycle 1, Day 15 | 0.106 | 0.7 | −9.4% | 5 |
| 12017 | baseline | 0.617 | 3.2 | | |
| 12017 | Cycle 1, Day 15 | 0.526 | 4.1 | −14.7% | 5 |
| 12017 | Cycle 2, Day 15 | 0.455 | 3.9 | −26.3% | 6 |
| 12019 | baseline | 0.498 | 3.0 | | |
| 12019 | Cycle 1, Day 15 | 0.434 | 5.0 | −12.9% | 5 |
| 12019 | Cycle 2, Day 15 | 0.400 | 0.6 | −19.7% | 6 |
| 12020 | baseline | 0.131 | 0.8 | | |
| 12020 | Cycle 1, Day 15 | 0.089 | 6.8 | −32.1% | 5 |
| 12020 | Cycle 2, Day 15 | 0.084 | 1.2 | −35.9% | 6 |
| 12021 | baseline | 0.017 | 17.7 | | |
| 12021 | Cycle 1, Day 15 | 0.015 | 1.4 | −11.8% | 5 |
| 12021 | Cycle 2, Day 15 | 0.013 | 9.9 | −23.5% | 6 |
| 12023 | baseline | 0.017 | 4.6 | | |
| 12023 | Cycle 1, Day 15 | 0.020 | 4.0 | 17.6% | 5 |
| 12024 | baseline | 2.700 | 4.7 | | |
| 12024 | Cycle 1, Day 15 | 1.970 | 4.5 | −27.0% | 5 |
| 12024 | Cycle 2, Day 15 | 1.710 | 7.3 | −36.7% | 6 |
| 12027 | baseline | 0.475 | 4.2 | | |
| 12027 | Cycle 1, Day 15 | 0.359 | 3.5 | −24.4% | 5 |
| 12027 | Cycle 2, Day 15 | 0.399 | 2.8 | −16.0% | 6 |
| 12029 | baseline | 0.918 | 0.7 | | |
| 12029 | Cycle 1, Day 15 | 0.676 | 7.3 | −26.4% | 5 |
| 12029 | Cycle 2, Day 15 | 0.610 | 0.4 | −33.6% | 6 |
| 12030 | baseline | 0.587 | 4.2 | | |
| 12030 | Cycle 1, Day 15 | 0.522 | 0.8 | −11.1% | 5 |
| 12030 | Cycle 2, Day 15 | 0.694 | 2.6 | 18.2% | 6 |
| 12031 | baseline | 0.920 | 1.9 | | |
| 12031 | Cycle 1, Day 15 | 0.756 | 1.9 | −17.8% | 5 |
| 12031 | Cycle 2, Day 15 | 0.466 | 1.9 | −49.3% | 6 |
| 12032 | baseline | 16.400 | 4.3 | | |
| 12032 | Cycle 1, Day 15 | 13.300 | 0.5 | −18.9% | 5 |
| 12032 | Cycle 2, Day 15 | 11.900 | 2.5 | −27.4% | 6 |
| 12034 | baseline | 0.764 | 1.1 | | |
| 12034 | Cycle 1, Day 15 | 0.568 | 2.3 | −25.7% | 5 |
| 12034 | Cycle 2, Day 15 | 0.338 | 5.6 | −55.8% | 6 |
| 12035 | baseline | 0.246 | 5.7 | | |
| 12035 | Cycle 1, Day 15 | 0.142 | 4.9 | −42.3% | 5 |
| 12036 | baseline | 0.163 | 2.7 | | |
| 12036 | Cycle 1, Day 15 | 0.288 | 3.4 | 76.7% | 5 |
| 12036 | Cycle 2, Day 15 | 0.083 | 14.6 | −49.1% | |
| 12037 | baseline | 0.116 | 4.4 | | |
| 12037 | Cycle 1, Day 15 | 0.113 | 2.4 | −2.6% | 5 |
| 12037 | Cycle 2, Day 15 | 0.040 | 51.0 | −65.5% | 6 |
| 12040 | baseline | 0.134 | 0.5 | | |
| 12040 | Cycle 2, Day 15 | 0.096 | 16.3 | −28.4% | 6 |
| 12041 | baseline | 0.091 | 4.7 | | |
| 12041 | Cycle 1, Day 15 | 0.111 | 9.5 | 22.0% | 5 |
| 12043 | baseline | 0.741 | 4.1 | | |
| 12043 | Cycle 1, Day 15 | 0.447 | 1.9 | −39.7% | 5 |
| 12043 | Cycle 2, Day 15 | 0.470 | 7.5 | −36.6% | 6 |
| 14003 | baseline | 1.440 | 5.8 | | |
| 14003 | Cycle 1, Day 15 | 0.778 | 1.3 | −46.0% | 5 |
| 14003 | Cycle 2, Day 15 | 0.918 | 4.0 | −36.3% | 6 |
| 14004 | baseline | 0.113 | 3.0 | | |
| 14004 | Cycle 1, Day 15 | 0.142 | 1.5 | 25.7% | 5 |
| 14004 | Cycle 2, Day 15 | 0.157 | 2.3 | 38.9% | 6 |
| 14005 | baseline | 0.101 | 3.7 | | |
| 14005 | Cycle 1, Day 15 | 0.086 | 5.0 | −14.9% | 5 |
| 14005 | Cycle 2, Day 15 | 0.078 | 0.0 | −22.8% | 6 |
| 14007 | baseline | 0.061 | 10.4 | | |
| 14007 | Cycle 1, Day 15 | 0.087 | 7.3 | 42.6% | 5 |
| 14007 | Cycle 2, Day 15 | 0.081 | 1.7 | 32.8% | 6 |
| 15001 | baseline | 0.911 | 2.0 | | |
| 15001 | Cycle 1, Day 15 | 0.769 | 4.9 | −15.6% | 5 |
| 15002 | baseline | 0.507 | 0.6 | | |
| 15002 | Cycle 1, Day 15 | 0.336 | 2.2 | −33.7% | 5 |
| 15002 | Cycle 2, Day 15 | 0.454 | 1.8 | −10.5% | 6 |
| 15005 | baseline | 0.202 | 0.8 | | |
| 15005 | Cycle 1, Day 15 | 0.320 | 3.1 | 58.4% | 5 |
| 15005 | Cycle 2, Day 15 | 0.077 | 6.6 | −61.9% | 6 |
| 15007 | baseline | 0.255 | 6.0 | | |
| 15007 | Cycle 1, Day 15 | 0.374 | 24.2 | 46.7% | 5 |
| 15007 | Cycle 2, Day 15 | 0.144 | 1.2 | −43.5% | 6 |
| 15008 | baseline | 0.596 | 1.4 | | |
| 15008 | Cycle 1, Day 15 | 0.590 | 2.0 | −1.0% | 5 |
| 16002 | baseline | 0.179 | 1.0 | | |
| 16002 | Cycle 1, Day 15 | 0.142 | 1.6 | −20.7% | 5 |
| 16002 | Cycle 2, Day 15 | 0.124 | 0.9 | −30.7% | 6 |
| 16003 | baseline | 0.018 | 2.1 | | |
| 16003 | Cycle 1, Day 15 | 0.012 | 3.1 | −37.5% | 5 |
| 16003 | Cycle 2, Day 15 | 0.012 | 27.1 | −34.8% | 6 |
| 16004 | baseline | 0.077 | 6.6 | | |
| 16004 | Cycle 1, Day 15 | 0.068 | 0.0 | −11.7% | 5 |
| 16004 | Cycle 2, Day 15 | 0.051 | 1.2 | −33.8% | 6 |
| 16006 | baseline | 0.079 | 0.5 | | |
| 16006 | Cycle 1, Day 15 | 0.041 | 6.6 | −48.1% | 5 |
| 16006 | Cycle 2, Day 15 | 0.029 | 23.0 | −63.3% | 6 |
| 17001 | baseline | 0.103 | 3.6 | | |
| 17001 | Cycle 1, Day 15 | 0.114 | 5.3 | 10.7% | 5 |
| 17004 | baseline | 0.635 | 2.2 | | |
| 17004 | Cycle 1, Day 15 | 0.439 | 0.0 | −30.9% | 5 |
| 17008 | baseline | 0.727 | 0.2 | | |
| 17008 | Cycle 1, Day 15 | 0.370 | 4.1 | −49.1% | 5 |
| 17008 | Cycle 2, Day 15 | 0.265 | 4.1 | −63.5% | 6 |
| 19005 | baseline | 1.190 | 1.6 | | |
| 19005 | Cycle 1, Day 15 | 1.390 | 2.4 | 16.8% | 5 |
| 19008 | baseline | 0.469 | 2.2 | | |
| 19008 | Cycle 1, Day 15 | 0.280 | 5.4 | −40.3% | 5 |
| 19010 | baseline | 0.055 | 3.1 | | |
| 19010 | Cycle 1, Day 15 | 0.044 | 7.8 | −20.0% | 5 |
| 19010 | Cycle 2, Day 15 | 0 036 | 2.0 | −34.5% | 6 |
| 19011 | baseline | 0.125 | 5.8 | | |
| 19011 | Cycle 1, Day 15 | 0.081 | 4.0 | −35.2% | 5 |
| 19011 | Cycle 2, Day 15 | 0.051 | 9.6 | −59.2% | 6 |
| 19012 | baseline | 0.093 | 8.0 | | |
| 19012 | Cycle 1, Day 15 | 0.080 | 16.1 | −14.0% | 5 |
| 19012 | Cycle 2, Day 15 | 0.047 | 1.0 | −49.5% | 6 |
| 22003 | baseline | 0.698 | 2.1 | | |
| 22003 | Cycle 1, Day 15 | 0.464 | 8.6 | −33.5% | 5 |
| 22003 | Cycle 2, Day 15 | 0.272 | 0.6 | −61.0% | 6 |
| 22004 | baseline | 1.640 | 0.3 | | |
| 22004 | Cycle 1, Day 15 | 0.783 | 2.9 | −52.3% | 5 |
| 22004 | Cycle 2, Day 15 | 0.534 | 3.0 | −67.4% | 6 |
| 22005 | baseline | 1.270 | 1.7 | | |
| 22005 | Cycle 1, Day 15 | 0.855 | 1.6 | −32.7% | 5 |
| 22005 | Cycle 2, Day 15 | 0.739 | 2.7 | −41.8% | 6 |
| 22006 | baseline | 1.740 | 2.2 | | |
| 22006 | Cycle 1, Day 15 | 1.420 | 3.6 | −18.4% | 5 |
| 22006 | Cycle 2, Day 15 | 1.480 | 4.0 | −14.9% | 6 |
| 22007 | baseline | 0.109 | 10.7 | | |
| 22007 | Cycle 1, Day 15 | 0.083 | 15.4 | −23.9% | 5 |
| 22007 | Cycle 2, Day 15 | 0.135 | 1.8 | 23.9% | 6 |
| 30001 | baseline | 0.095 | 0.5 | | |
| 30001 | Cycle 1, Day 15 | 0.092 | 0.0 | −3.2% | 5 |
| 30001 | Cycle 2, Day 15 | 0.157 | 0.8 | 65.3% | 6 |
| 30002 | baseline | 0.714 | 4.2 | | |
| 30002 | Cycle 1, Day 15 | 0.279 | 4.3 | −60.9% | 5 |
| 30002 | Cycle 2, Day 15 | 0.156 | 0.9 | −78.2% | 6 |
| 30003 | baseline | 0.157 | 0.3 | | |
| 30003 | Cycle 1, Day 15 | 0.158 | 3.0 | 0.6% | 5 |
| 30003 | Cycle 2, Day 15 | 0.131 | 1.8 | −16.6% | 6 |
| 30004 | baseline | 0.102 | 1.7 | | |
| 30004 | Cycle 1, Day 15 | 0.088 | 3.5 | −13.7% | 5 |
| 30004 | Cycle 2, Day 15 | 0.089 | 0.3 | −12.7% | 6 |

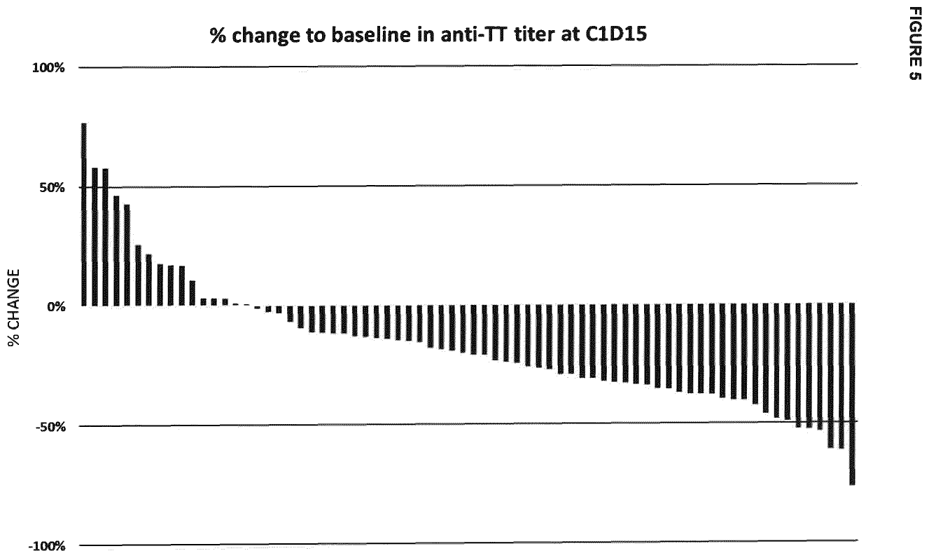

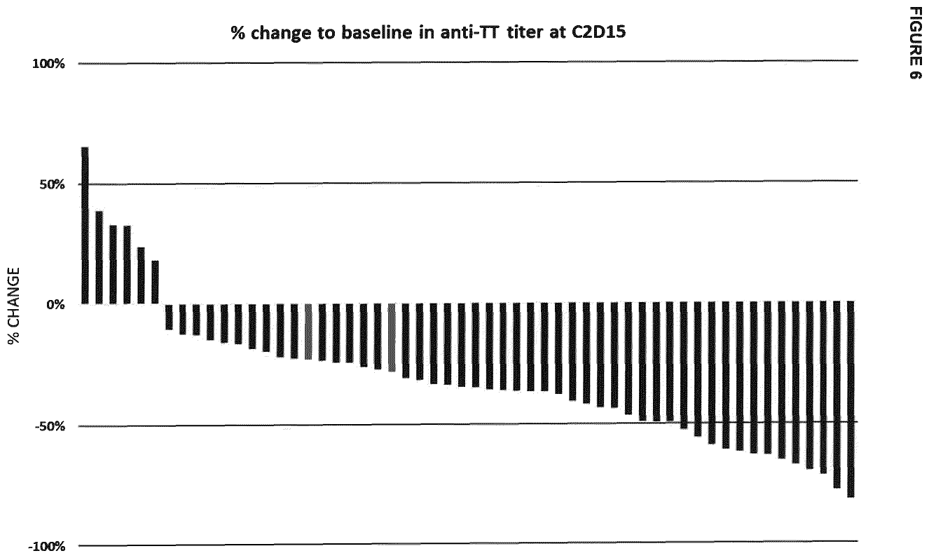

To determine the impact of MOR202 on anti-TT antibody titers, serum samples obtained at day 0 (prior to MOR202 treatment, indicated as “baseline” in Table 4), day 15 (cycle 1) and day 43 (=cycle 2, day 15) after MOR202 administration were analyzed. At day 15 of cycle 1, following MOR202 treatment, the majority of subjects showed a significant reduction of anti-TT antibody titers compared to baseline at day 0. The % change of anti-TT concentrations of “baseline” samples obtained at day 0 compared to samples obtained at day 15 of cycle 1 (indicated as “Cycle 1, Day 15”) are shown in FIG. 5. The % change of anti-TT concentrations of “baseline” samples obtained at day 0 compared to samples obtained at day 15 of cycle 2 (indicated as “Cycle 2, Day 15”) are shown in FIG. 6. In the majority of MOR202 treated subjects anti-TT antibody titers further decreased (i.e. higher percentage change from cycle 1, day 15 to cycle 2, day 15) suggesting a long-term effect of MOR202 on antibody titers.

In summary, these data demonstrate that MOR202 is effective in the reduction of serum antibody titers. Therefore, an effective treatment and/or prophylaxis of autoantibody-mediated AD using an anti-CD38 antibody (e.g. MOR202) is highly plausible.

### Example 2: Determination of M-Protein Levels

### 2.1. Study Design

M-Protein levels in serum samples of multiple myeloma patients (enrolled in the trial of Example 1) were quantitatively determined by capillary electrophoresis (CE) assays, in particular serum protein electrophoresis (SPEP) and urine protein electrophoresis (UPEP).

### 2.2. Capillary Electrophoresis—Principle of Test

Charged molecules are separated by their electrophoretic mobility at a specific pH in an alkaline buffer. Separation occurs according to the electrolyte pH and electroosmotic flow. Each sample is diluted in a dilution buffer and the capillaries are filled with the separation buffer; samples are then injected by aspiration into the anodic end of the capillary. This is followed by high voltage protein separation. Subsequently, direct detection and quantification of the different protein fractions is performed at a specific wavelength at the cathodic end of the capillary.

Further assays for the assessment of M-Protein levels include, but are not limited to immunofixation electrophoresis (IFE), serum free light chain (sFLC) assay and total protein determination (Keren D F and Schroeder L, Clin Chem Lab Med. 2016 Jun. 1; 54(6):947-61). In addition, an IFE-based REFELX assay as described in WO/2017/149122 can be performed.

### 2.3 Results:

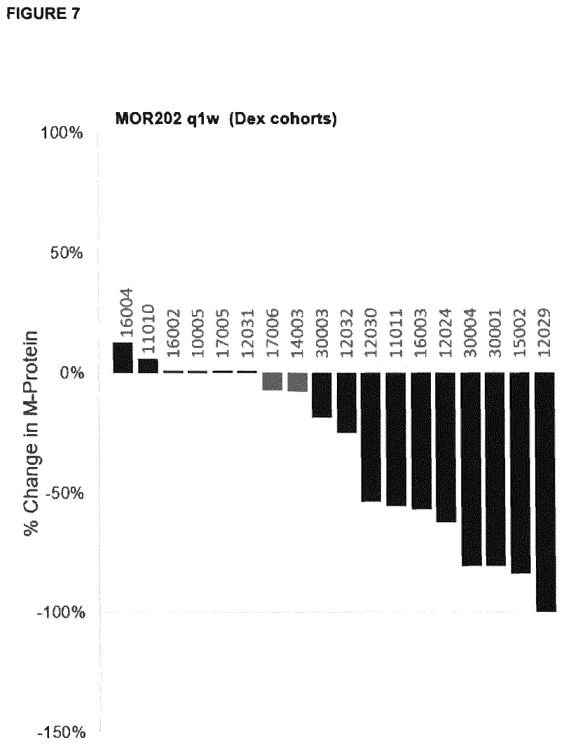

FIG. 7 shows the change given as percentage [%] of M-Protein levels in multiple myeloma patients after MOR202 treatment.

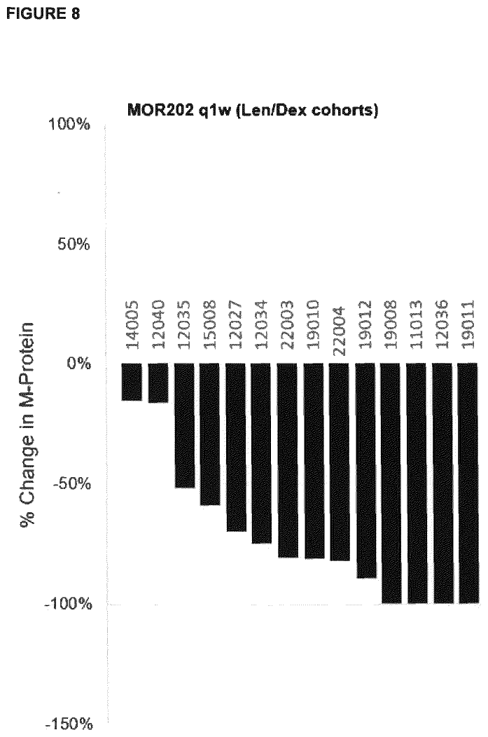

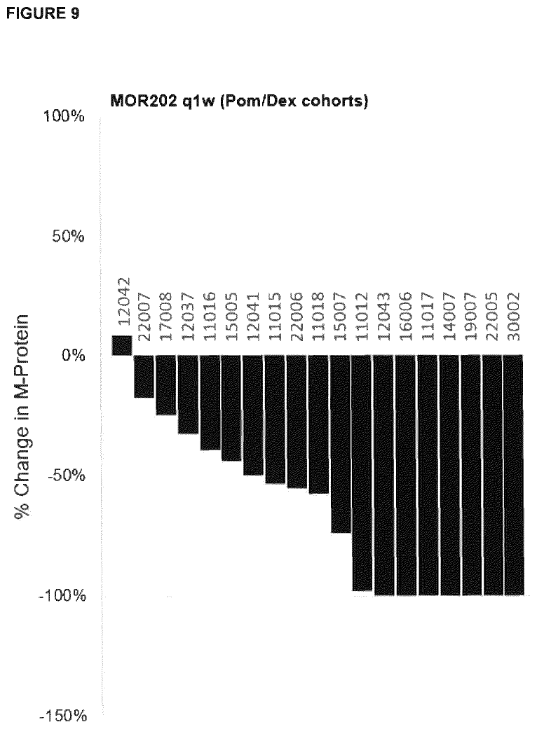

The effect of MOR202 in decreasing M-Protein indicates indirectly the destruction and depletion of M-Protein producing malignant plasma cells. In addition to the results of example 1, shown in FIGS. 5 and 6, the decrease of M-Protein after MOR202 administration (FIGS. 7 to 9) provides further evidence that MOR202 is effective in reducing antibody titers.

### Example 3: Evaluation of ADCC Mediated by Natural Killer (NK) Cells

### 3.1 Experimental Setup

To test the specific killing effect of MOR202, Daratumumab and Isatuximab (SAR650984) mediated by natural killer cells on (i) a CD38 high expressing multiple myeloma cell line (NCI-H929) and (ii) CD38 low expressing human NK cells an ADCC assay was performed. NK cells where purified from human blood by MACS (Miltenyi Biotec, Cat No.: 130-092-657). NK cell purity was evaluated by FACS using the CD3/CD16+CD56/CD45 Tritest™ (Becton Dickinson Cat No.: 342411). NCI-H929 target cells were incubated with the respective antibody at defined concentrations and an effectortarget cell ratio of 3:1 for 2-4h at 37° C. NK target cells were incubated for 2-4h at 37° C. with the respective antibody only, as for the NK cell:NK cell setup, target and effector cells are the same. To determine cytotoxicity, propidium iodide (PI) was added to the cell sample after incubation and PI uptake into dead cells was immediately assessed by flow cytometry.

### 3.2 Results

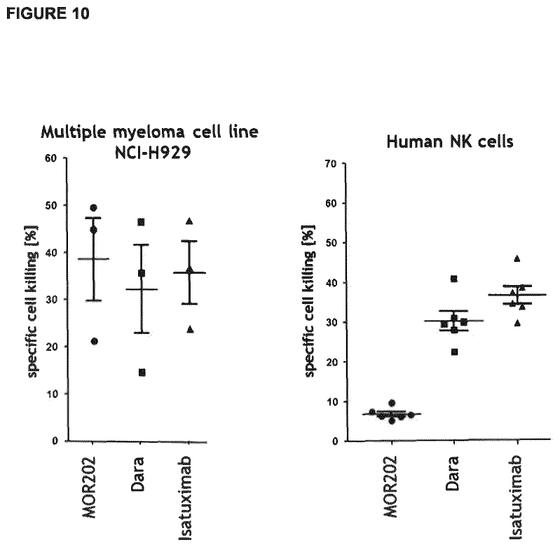

The results of specific cell killing [%] for MOR202, Daratumumab and Isatuximab on NCI-929 and NK cells are shown in FIG. 10.

### Example 4: Evaluation of Safety and Efficacy of MOR202 in Subjects with Anti-PLA2R Positive Membranous Nephropathy (aMN)

### 4.1 Study Design

Objectives of the study are to evaluate the safety, tolerability and efficacy of the human anti-CD38 antibody MOR202 in patients with anti-PLA2R positive membranous nephropathy (aMN) and to assess the effect of MOR202 on serum anti-PLA2R antibodies levels.

MOR202 dosing is based on the results of the clinical study of Example 1 in multiple myeloma (MM) as well as a PK/PD modelling approach. There, MOR202 was administered in a dose escalating scheme at 0.1 to 16 mg/kg i.v. once weekly (QW) or every two weeks (Q2W) incl. a loading dose on Cycle 1 Day 4. MOR202 was applied either as a single agent (monotherapy) or in combination with DEX, POM/DEX or LEN/DEX. The overall treatment duration was based on the clinical response with a continuous treatment for up to 3 years at a maximum. With the results a population based PK/PD model was established considering the different target expression rate between MM and aMN subjects. The model was used to simulate drug exposure as expected in this study (i.e. dosing at 16 mg/kg: 4×QW followed by 5×Q4W) and results were compared to the data of the study of Example 1 considering the same treatment period. 6 patients were dosed for at least 24 weeks in the study of Example 1 at 16 mg/kg QW incl. a loading dose at Day 4. This should lead to a 2.4-fold excess in MOR202 exposure compared to the anticipated dose and dosing regimen in the current study with similar maximum serum concentrations. The purpose of the trial is to evaluate the safety and efficacy of the human anti-CD38 antibody MOR202 in patients with anti-PLA2R positive membranous nephropathy (aMN) eligible for immunosuppressive therapy for the first time or who have failed to respond to immunosuppressive therapy (IST), including rituximab (anti-CD20) therapy.

### Example 5: M-PLACE: A Phase Ib/IIa Multicenter Open-Label Study for Treatment of Two Cohorts of aMN Patients with MOR202 (NCT04145440)

A phase Ib/IIa, open-label, multicenter clinical trial to assess safety and efficacy of the human anti-CD38 antibody MOR202 in anti-PLA2R antibody positive membranous nephropathy (aMN) with an estimated enrollment of 30 participants has been initiated and is recruiting in at least 14 centers at 6 locations in the US and Europe. ClinicalTrials.gov identifier (NCT number): NCT04145440.

### 5.1. Study Design

Objectives of the study are to evaluate the safety, tolerability and efficacy of the human anti-CD38 antibody MOR202 in patients with anti-PLA2R positive membranous nephropathy (aMN) and to assess the effect of MOR202 on serum anti-PLA2R antibodies levels.

The main treatment rationale is the reduction of membranous nephropathy (MN) disease specific anti-PLA2R antibodies through targeted depletion of autoantibody producing plasma cells by anti-CD38 antibody MOR202.

The patient population to be treated includes adult subjects with biopsy-proven MN positive for anti-PLA2R antibodies. Ages eligible for study: 18 to 80 years (adults, older adults). All sexes are eligible for study.

### Key Inclusion Criteria:

Urine protein to creatinine ratio of ≥3.0 g/g (as measured from a 24 h urine collection)
Estimated glomerular filtration rate ≥50 mL/min/1.73 m² or >30 and <50 mL/min/1.73 m², and interstitial fibrosis and tubular atrophy score of less than 25% on a renal biopsy obtained within the last 6 months prior to start of screening.
on supportive treatment with an Angiotensin Converting Enzyme Inhibitor or an Angiotensin II Receptor Blocker for at least 4 weeks prior to Screening, having reached a stable dose.
Systolic BP≤150 mmHg and diastolic BP≤100 mmHg
Vaccinated against Pneumococcus within the last 3 years prior to date of signing informed consent (subjects may be vaccinated during screening to meet this criterion; interval to first dose of MOR202 must be at least 14 days).
Cohort 1a (newly diagnosed patients): Serum anti-PLA2R antibodies ≥150.0 Response Units (RU)/mL determined at screening by Euroimmun ELISA.
Cohort 1b, relapse subjects: Must have had complete immunological and/or clinical remission according to judgement of the investigator and serum anti-PLA2R antibodies ≥50.0 RU/mL determined at screening by Euroimmun ELISA.
Cohort 2: Failure of previous therapy, i.e. subject never achieved a complete immunological and/or clinical remission according to judgement of the investigator during or after completion of a recognized IST containing cyclosporine A, tacrolimus, mycophenolate-mofetil, ACTH or alkylating agents (e.g. cyclophosphamide), or rituximab. Serum anti-PLA2R antibodies ≥20.0 RU/mL determined at screening by the Euroimmun ELISA.

### Key Exclusion Criteria:

Hemoglobin <90 g/L.
Thrombocytopenia: Platelets <100.0×10⁹/L.
Neutropenia: Neutrophils <1.5×10⁹/L.
Leukopenia: Leukocytes <3.0×10⁹/L.
Hypogammaglobulinemia: Serum immunoglobulins <5.0 g/L.
Secondary cause of MN (e.g. systemic lupus erythematosus, medications, malignancies)
Concomitant renal disease other than MN (e.g., diabetic renal disease, lupus nephritis, IgA nephropathy).

Cohort 1 comprises approximately 20 aMN patients stable on supportive care treatment with ACEI/ARB at screening with unfavourable prognostic features such as proteinuria (>5 g/24h) and high and stable serum titers of anti-PLA2R antibodies (≥150.00 response units (RU)/mL, EuroImmun ELISA) eligible for IST, or subjects relapsing after complete or partial proteinuria response including a serum anti-PLA2R antibody titer less than 20 RU/mL for at least 6 months. Subjects may be newly diagnosed (Cohort 1a) or relapsing (Cohort 1b) after a prior proteinuria and immunological response to IST.

Cohort 2 comprises approximately 10 aMN patients requiring 2nd or 3rd line IST who did not respond immunologically to their last prior line of therapy and thus are considered refractory. Failure of previous therapy, i.e. subject never achieved a reduction of serum anti-PLA2R antibody titers to below 20 RU/mL during or after completion of a recognized IST containing CSA, tacrolimus, MMF, ACTH or alkylating agents (e.g. cyclophosphamide), or rituximab determined after at least 6 months after start of therapy.

Exclusion criteria for both cohort 1 and cohort 2 are active infection, secondary cause of MN (e.g. SLE, medications, malignancies), Type 1 or 2 diabetes mellitus, pregnancy or breast feeding, known or suspected hypersensitivity to the study drugs and its excipients.

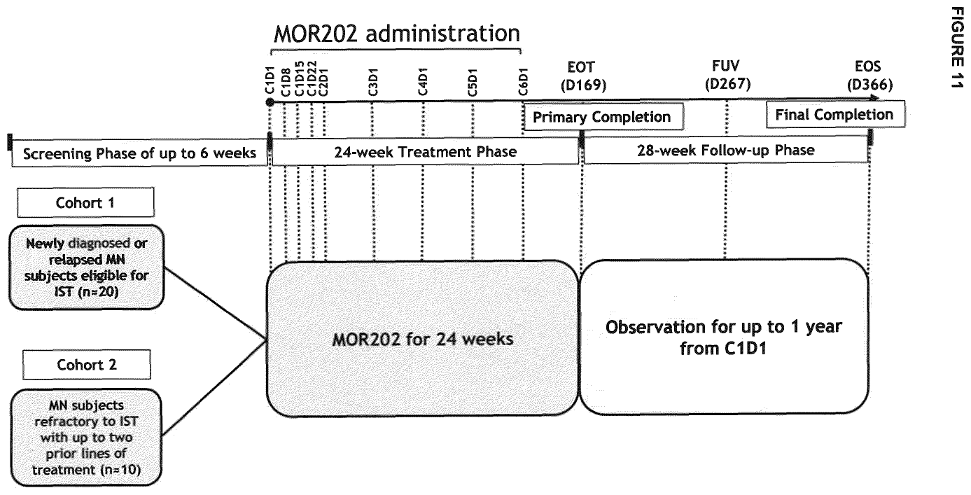

MOR202 monotherapy treatment of the two cohorts is over a 24-week treatment phase followed by a 28-week observational follow-up phase (FIG. 11).

### 5.2. Administration of MOR202 (MOR03087)

MOR202 is supplied as a lyophilized powder for reconstitution in labelled glass vials. MOR202 must be stored at 2-8° C. until use. For drug preparation each vial must be reconstituted with 4.8 mL water for injection (WFI). After reconstitution each vial contains 325 mg of MOR202 (MOR03087) in an extractable volume of 5 mL (65 mg/mL). For infusion it will be diluted in 250 mL 0.9% sodium chloride solution.

All subjects will be treated for 24 weeks distributed to six 28-day treatment cycles. In total, 9 doses of MOR202 will be administered on the following treatment days: Cycle 1 day 1, 8, 15 and 22, and on day 1 of cycles 2-6 (FIG. 11). In the first treatment cycle, MOR202 will be administered at 16 mg/kg once weekly (i.e. 4 doses for cycle 1 in total). In treatment cycles 2-6, MOR202 will be administered at 16 mg/kg once every 4 weeks at the first day of each cycle (i.e. C2D1, C3D1, . . . ; 5 doses for cycles 2-6 in total).

The first MOR202 i.v. infusion shall be slow (approximately 90 minutes, about 3 mL/min). If no infusion reactions occur, the infusion time may be shortened to 1 hour or shorter in subsequent infusions but limited to the shortening steps outlined in Table 5. Infusion time should not be shorter than 30 minutes. Premedication of subjects with antihistamines and antipyretic drugs (e.g. paracetamol/acetaminophen) as prophylaxis of infusion related reactions (IRRs) is recommended. Co-medication for prevention of IRRs with i.v. dexamethasone (or equivalent glucocorticoids administered i.v.) approximately 30 minutes before start of MOR202 infusion is mandatory for the first 3 applications as outlined in Table 5.

TABLE 5
MOR202 infusion guideline

| | | | | |
|---|---|---|---|---|
| MOR202 infusion number | 1 | 2 | 3 | 4th and |
| | | | | onwards |
| Minimum infusion time | 90 min | 60 min | 30 min | 30 min |
| Maximum infusion rate | 3 mL/min | 4.5 mL/min | 9 mL/min | 9 mL/min |
| Dexamethasone i.v. dose | 16 mg | 16 mg | 8 mg | not |
| | | | | mandatory |

### 5.3. Assessment of Safety, Immunogenicity and Pharmacokinetics

Safety will be assessed in terms of physical examination, vital signs, oxygen saturation, electrocardiograms, hematological and biochemical tests, adverse events and immunogenicity. Adverse events will be graded according to NCI CTCAE, version 4.03. To monitor for immunogenicity and pharmacokinetics the presence of anti-MOR202 antibodies (anti-drug antibodies) and serum concentrations of MOR202, respectively at selected time points will be assessed during the course of the study.

### 5.4. Efficacy Assessments

Major efficacy assessments include: (i) Serum anti-PLA2R antibody levels measured by ELISA to track the course of immunological response before, during and after MOR202 therapy. (ii) Proteinuria based on UPCR from 24h urine/spot urine measured during and after MOR202 therapy. (iii) Kidney function determined before, during and after MOR202 therapy by estimating glomerular filtration rate (eGFR) based on the CKD-epi equation. (iV) Urinary Sodium excretion determined from 24h urine.

### 5.5. Biomarker

Presence and titer of anti-PLA2R antibodies at selected time points (i.e. kinetics of anti-PLA2R antibody titers) will be determined for all subjects during the course of the study. Optionally, additional autoantibody titers (e.g., anti-thrombospondin type-1 domain-containing 7A, anti-THSD7A), anti-tetanus toxoid and/or anti-EBV antibodies at selected time points can be monitored. Serum concentrations of total IgG, IgA and IgM can be assessed by ELISA. Quantitative NK cell, B cell, T cell (incl. regulatory T cell), plasma blast, plasma cell numbers at selected time points can be determined by peripheral blood flow cytometry or ELISPOT assays.

### 5.6. KDQOL-36

The Kidney Disease Quality of Life (KDQOL-36™) survey is used for the assessment of the Quality of Life (QoL) defined as score change from baseline in patients with autoimmune membranous nephropathy treated with MOR202.

### Example 6: Determination of Anti-PLA2R Antibody Levels

Anti-phospholipase A2 receptor (PLA2R) antibody levels in human serum samples will be determined quantitatively by monospecific ELISA (enzyme immunoassay with a single antigen, Euroimmune, Order No. EA 1254-G) according to the manufactures instructions. In brief, polystyrene microplate strips coated with purified, PLA2R antigens are used as solid phase. Serum dilutions 1:101 will be prepared and incubated on the antigen bound to wells of the microplate. If the sample is positive, specific antibodies in the diluted serum sample attach to the PLA2R antigen coupled to the solid phase. Unbound antibodies are washed away and in a further step, the attached anti-PLA2R specific antibodies are detected with peroxidase-labelled anti-human IgG. Bound antibodies are made visible using a chromogen/substrate solution, which is capable of promoting a colour reaction. The intensity of the colour produced is proportional to the concentration of antibodies in the serum sample.

1: A method for treatment and/or prophylaxis of autoantibody-mediated membranous nephropathy in a subject comprising administering to the subject an antibody or antibody fragment specific for CD38.

2: The method of claim 1, wherein the autoantibody-mediated membranous nephropathy is an anti-PLA2R and/or anti-THSD7A positive membranous nephropathy.

3: The method of claim 1, wherein the antibody or antibody fragment depletes plasma cells by antibody-dependent cell-mediated cytotoxicity (ADCC) and/or antibody-dependent cell-mediated phagocytosis (ADCP).

4: The method of claim 1, wherein the antibody shows a significant higher specific cell killing on plasma cells than on CD38 low expressing cells (e.g. NK cells).

5: The method of claim 1, wherein antibody administration leads to a reduction of endogenous autoantibody titers.

6: The method of claim 5, wherein said endogenous autoantibody titers comprise anti-PLA2R and/or anti-THSD7A autoantibodies.

7: The method of claim 1, wherein said antibody or antibody fragment specific for CD38 is a human antibody.

8: The method of claim 1, wherein said antibody or antibody fragment specific for CD38 is an IgG1.

9: The method of claim 1, wherein the antibody or antibody fragment comprises a HCDR1 region of amino acid sequence SEQ ID NO.: 1, a HCDR2 region of amino acid sequence SEQ ID NO.: 2, a HCDR3 region of amino acid sequence SEQ ID NO.: 3, and a LCDR1 region of amino acid sequence SEQ ID NO.: 4, a LCDR2 region of amino acid sequence SEQ ID NO.: 5 and a LCDR3 region of amino acid sequence SEQ ID NO.: 6.

10: The method of claim 1, wherein said antibody or antibody fragment specific for CD38 comprises a variable heavy chain region of SEQ ID NO.: 7 and a variable light chain region of SEQ ID NO.: 8.

11: The method of claim 1, wherein said antibody or antibody fragment is administered in combination with a further therapeutic agent.

12: The method of claim 11, wherein the further therapeutic agent Is an agent for the prophylaxis and/or treatment of autoantibody-mediated autoimmune disease.

13: The method of claim 12, wherein the further therapeutic agent is an immunosuppressive drug such as dexamethasone, azathioprine, mycophenolic acid, methotrexate or a proteasome inhibitor such as bortezomib.

14: The method of claim 1, wherein the antibody or antibody fragment is administered intravenously.

15: The method of claim 1, wherein the antibody or antibody fragment will be administered in the first treatment cycle at 16 mg/kg once weekly.

16: A pharmaceutical composition comprising an antibody or antibody fragment specific for CD38 in an amount effective to treat or prevent autoantibody-mediated autoimmune disease (AD) or symptoms associated with AD and a pharmaceutically acceptable carrier, diluent or excipient.

17: The pharmaceutical composition of claim 16, wherein said antibody or antibody fragment depletes plasma cells by antibody-dependent cell-mediated cytotoxicity (ADCC) and/or antibody-dependent cell-mediated phagocytosis (ADCP).

18: The pharmaceutical composition of claim 16, wherein said antibody or antibody fragment comprises a HCDR1 region of amino acid sequence SEQ ID NO.: 1, a HCDR2 region of amino acid sequence SEQ ID NO.: 2, a HCDR3 region of amino acid sequence SEQ ID NO.: 3, and a LCDR1 region of amino acid sequence SEQ ID NO.: 4, a LCDR2 region of amino acid sequence SEQ ID NO.: 5 and a LCDR3 region of amino acid sequence SEQ ID NO.: 6.

19: The pharmaceutical composition of claim 16, wherein said antibody or antibody fragment specific for CD38 comprises a variable heavy chain region of SEQ ID NO.: 7 and a variable light chain region of SEQ ID NO.: 8.

20: The pharmaceutical composition of claim 16 further comprising a further therapeutic agent for the prophylaxis and/or treatment of autoantibody-mediated autoimmune disease.
